(19) United States
(12) Patent Application Publication
(10) Pub. No.: US 2012/0266324 A1
(43) Pub. Date: Oct. 18, 2012

(54) Self-Reproducing Hybrid Plants

(75) Inventor: SHAI J. LAWIT, URBANDALE, IA (US)

(73) Assignee: PIONEER HI BRED INTERNATIONAL INC., JOHNSTON, IA (US)

(21) Appl. No.: 13/445,419

(22) Filed: Apr. 12, 2012

Related U.S. Application Data
(60) Provisional application No. 61/475,947, filed on Apr. 15, 2011.

(51) Int. Cl. A01H 1/02; C12N 15/63; C07H 21/04; A01H 5/00; A01H 5/10

(52) U.S. Cl. 800/275; 800/260; 800/298; 536/24.5; 800/306; 800/322; 800/314; 800/317.3; 800/312; 800/320.1; 800/320.3; 800/320; 800/320.2; 435/320.1

(57) ABSTRACT

Compositions and methods for the production of self-reproducing hybrid plants are provided. Compositions include suppression cassettes encoding polynucleotides and promoters that result in the MiMe diploid gamete phenotype compositions and suppression cassettes and expression cassettes useful for genome elimination of a parental diploid gamete in a fertilized zygote. The methods involve crossing a first plant comprising a first suppression cassette responsible for producing the MiMe diploid gamete phenotype and a first expression cassette expressing an active CENH3 mutant with a second plant comprising a second suppression cassette that reduces the level of wild-type CENH3 and a second expression cassette comprising a polynucleotide expressing CENH3 specifically in the ovule. Self fertilization of the resultant progeny plant results in the elimination of the female diploid genome in the zygote and normal development of the endosperm. Additionally provided are plants and seeds produced by the methods of the invention.

### CROSS-REFERENCE TO RELATED APPLICATIONS

This is a utility application which claims priority of Provisional Application Ser. No. 61/475,947 filed Apr. 14, 2011, hereby incorporated by reference in its entirety.

### FIELD

The invention relates to the field of genetic manipulation of plants, particularly the production of self-reproducing hybrid plants.

### BACKGROUND

Although plant breeding programs worldwide have made considerable progress developing new cultivars with improved disease resistances, yields and other useful traits, breeding as a whole relies on screening numerous plants to identify novel, desirable characteristics. Very large numbers of progeny from crosses often must be grown and evaluated over several years in order to select one or a few plants with a desired combination of traits.

A continuing goal of plant breeders is to develop stable, high-yielding varieties that are agronomically sound. Standard breeding of diploid plants often requires screening and back-crossing of a large number of plants to achieve the desired genotype. One solution to the problem of screening large numbers of progeny has been to generate doubled haploid plants that eliminate genomic heterogeneity and, thus, any segregation of traits. When economically and biologically feasible, additional gains are often made through employing heterosis with hybrids of two inbred parents.

Heterosis studies in soybean estimate that there is approximately a 10% yield improvement potential with hybrids. However, hybrid soybeans have never been developed because pollen flow from male to female inbreds is very poor. Pollen vectoring is a problem that has few, if any, solutions available for high volume hybrid production in soybean. However, hand crosses could produce limited hybrid numbers and volume production of hybrid soybean could commence with the aid of self-reproduction.

Furthermore, current transgene introgression requires the maintenance of transgene homozygosity in inbred lines and varieties, which greatly limits the potential for native and transgene trait stacking. However, by using hybrid plants, transgenes could be stacked much more easily by providing a single copy from each parent. Availability of a system to generate self-reproducing hybrids would find value in both plant breeding and development.

Thus, marked improvements in the economics of breeding can be achieved via self-reproducing hybrid production, since selection and other procedural efficiencies can be substantially improved. Current methods for parent-specific genome elimination result in plants with near total male sterility and very low rates of female fecundity, making propagation of the hybrid plant difficult.

### BRIEF SUMMARY

Compositions and methods for the production of self-reproducing hybrid plants are provided. Compositions include suppression cassettes encoding polynucleotides and promoters that result in the MiMe diploid gamete phenotype. Further provided are methods and compositions comprising suppression cassettes and expression cassettes resulting in genome elimination of a parental diploid gamete in the fertilized zygote, producing a self-reproducing hybrid plant.

Methods for producing a self-reproducing hybrid plant include crossing a first plant comprising a first suppression cassette responsible for producing the MiMe diploid gamete phenotype and a first expression cassette expressing an active CENH3 mutant with a second plant comprising a second suppression cassette that reduces the level of wild-type CENH3 and a second expression cassette comprising a polynucleotide expressing CENH3 specifically in the ovule. Self fertilization of the resultant progeny plant results in the elimination of the female diploid genome in the zygote and normal development of the endosperm. Additionally provided are plants and seeds, particularly hybrid plants and hybrid seeds, produced by the methods of the invention.

### BRIEF DESCRIPTION OF THE DRAWINGS

This patent or application file contains at least one drawing figure executed in color. Copies of this patent or patent application publication with color drawing(s) will be provided by the Office upon request and payment of the necessary fee.

FIG. 1 shows the transgene system designed to activate clonal reproduction in hybrids, but maintain normal sexual reproduction in the parental inbred varieties.

FIG. 2 shows an example of the transgene system designed to activate clonal reproduction in hybrids, but maintain normal sexual reproduction in the parental inbred varieties. T7 polymerase and Gal4DBD-VP16 (or LexA) two component activation systems are shows as examples of possible transactivators that would activate the self reproduction system only once brought together in a hybrid cross containing the two transgene cassettes where the amiRNA silencing elements would be activated.

FIG. 3 shows the mechanisms utilized to result in self-reproducing hybrid plants.

FIG. 4 shows (Left) quadruply labeled embryo sac in an ovule from Arabidopsis transgenic PHP47078 at the egg cell stage of development. These labeled embryo sac cells allow cell development and viability to be monitored. (Right) Triply labeled embryo sac in an ovule from Arabidopsis transgenic PHP42551. This embryo sac is at the early embryo stage of development prior to the globular stage. Numerous endosperm nuclei are visible in cyan demonstrating the ability to follow early endosperm development.

FIG. 5. PHP51198 T1 microspores (5A) and T2 root cell squashes (5B) demonstrating MiMe phenotypes. The MiMe phenotypes displayed are dyad microspore development instead of quartet, and tetraploidy in the T2 generation instead of diploidy.

FIG. 6. Alternate Strategy for 4593—Self Reproducing Hybrid Carrying Cassettes and 2.

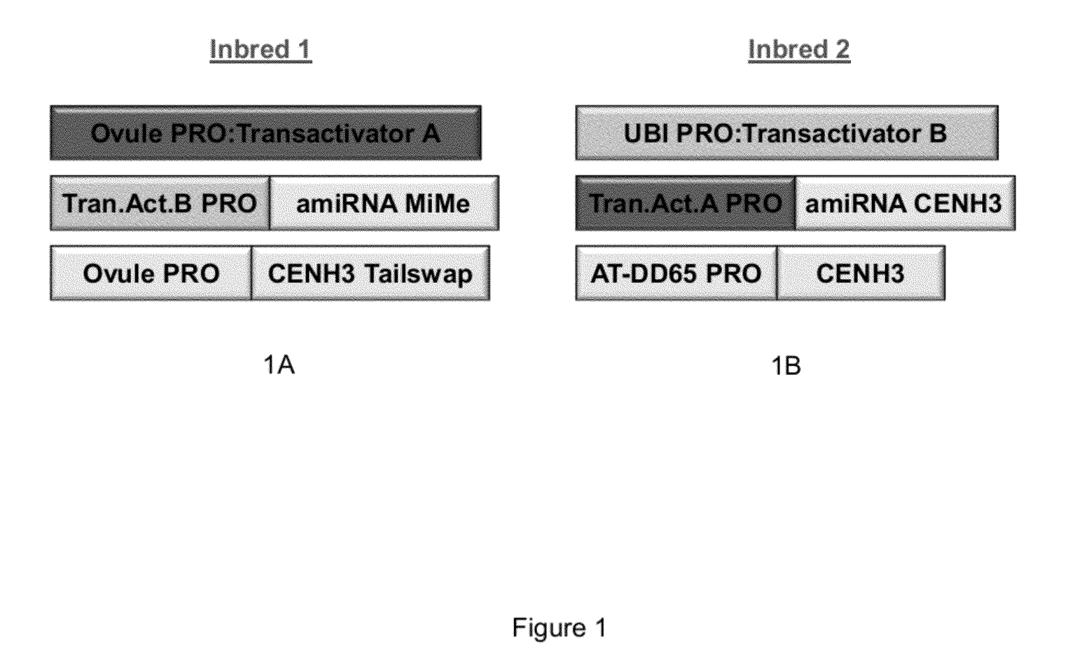

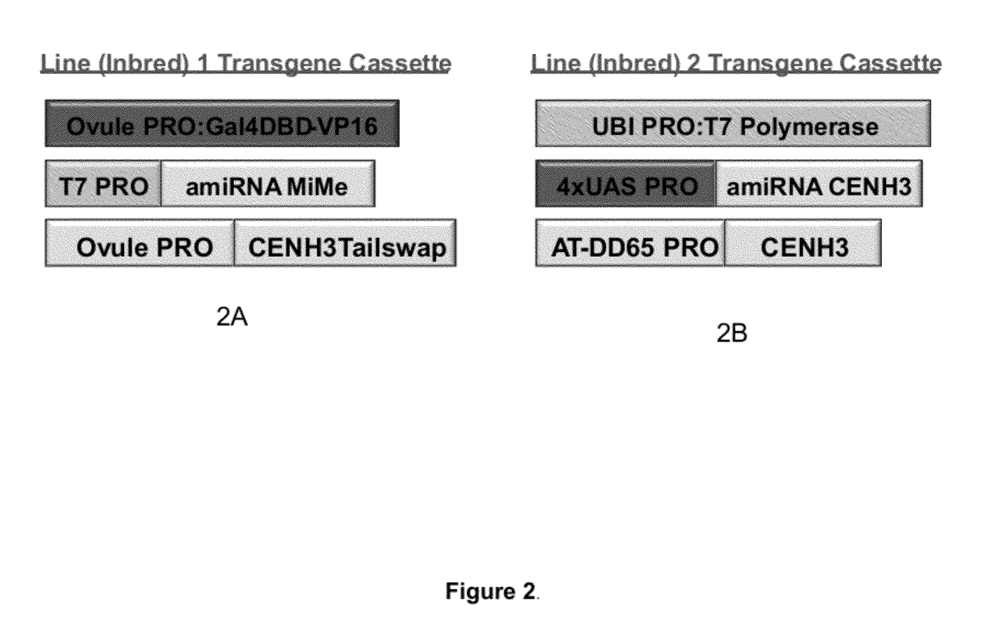

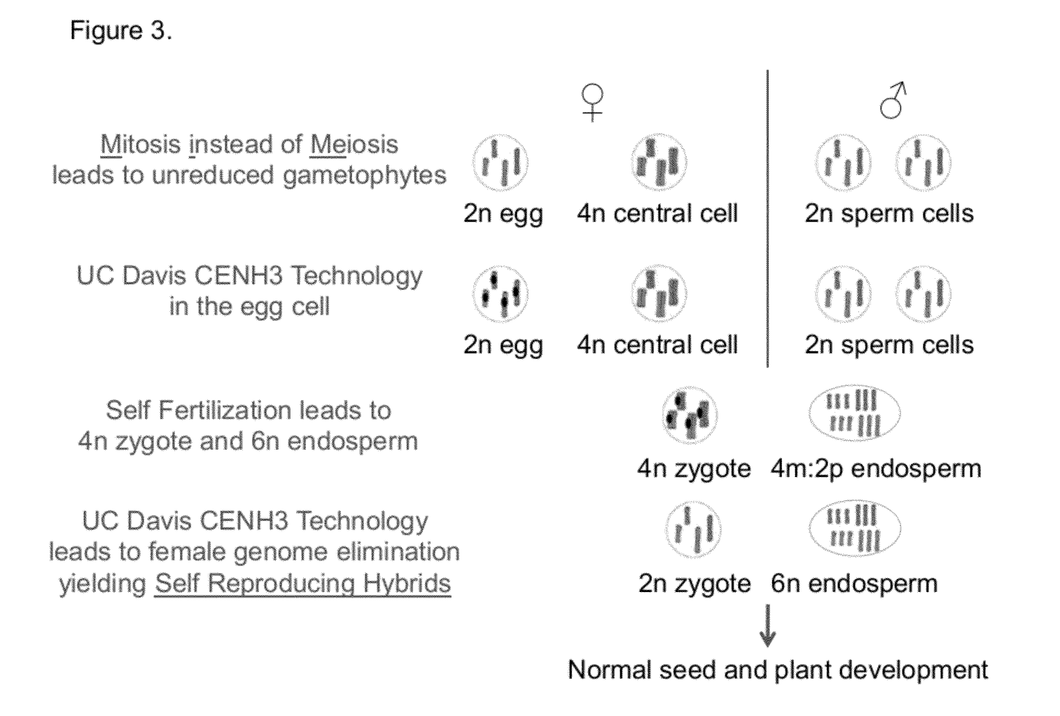

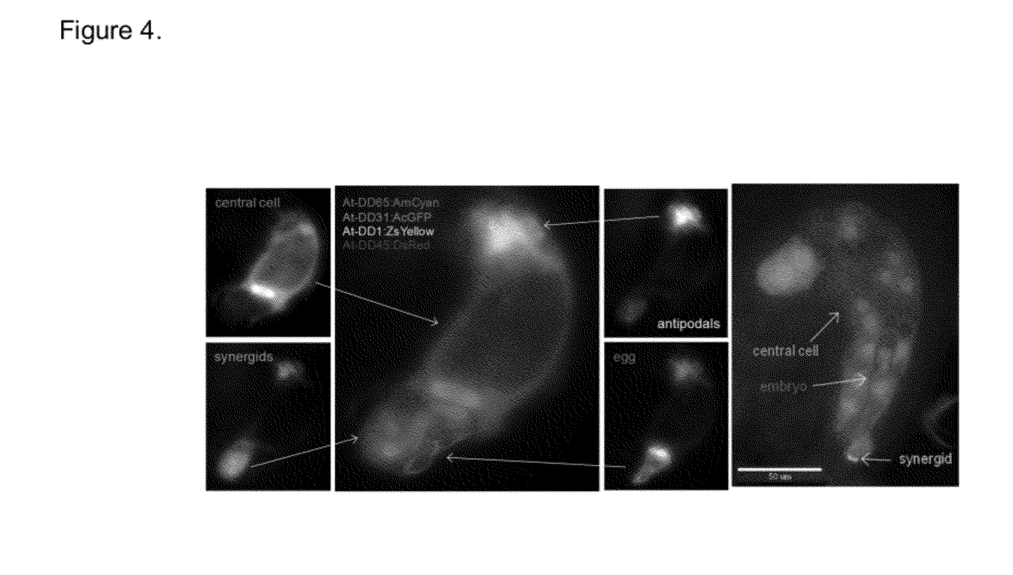

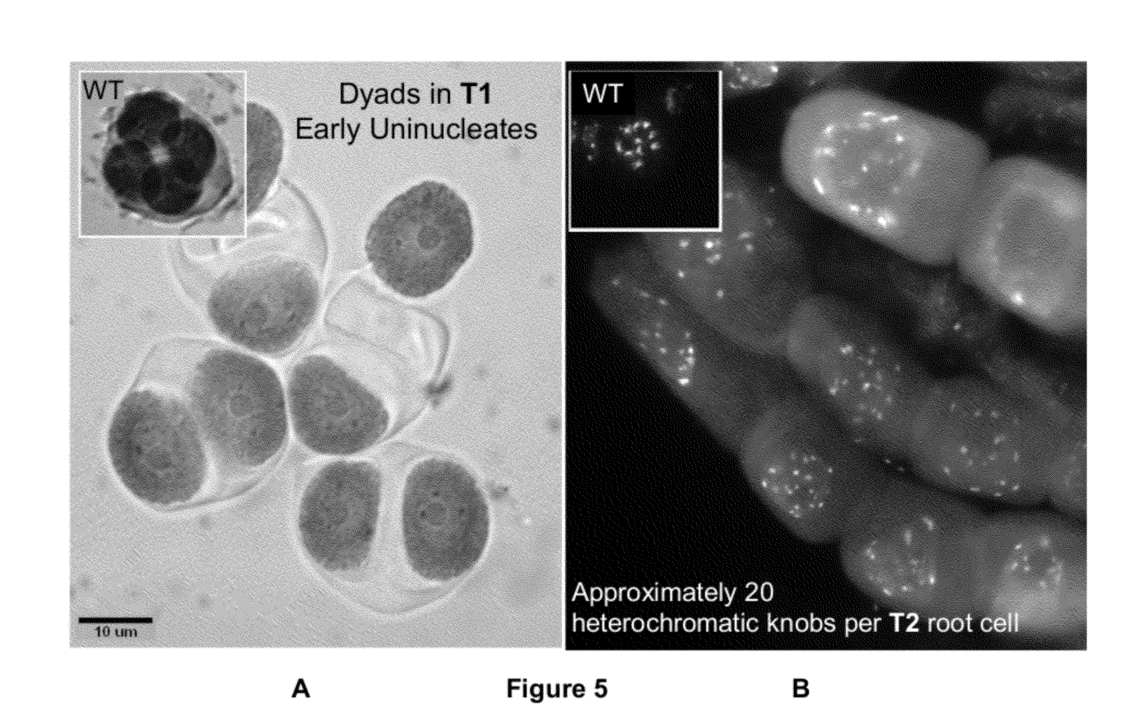

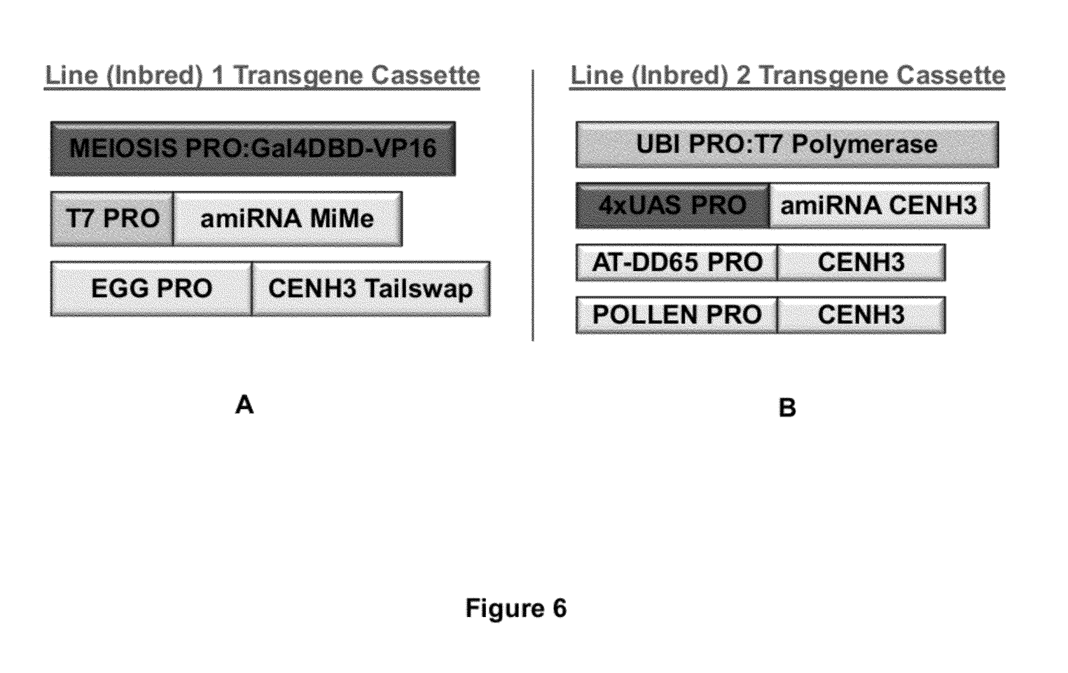

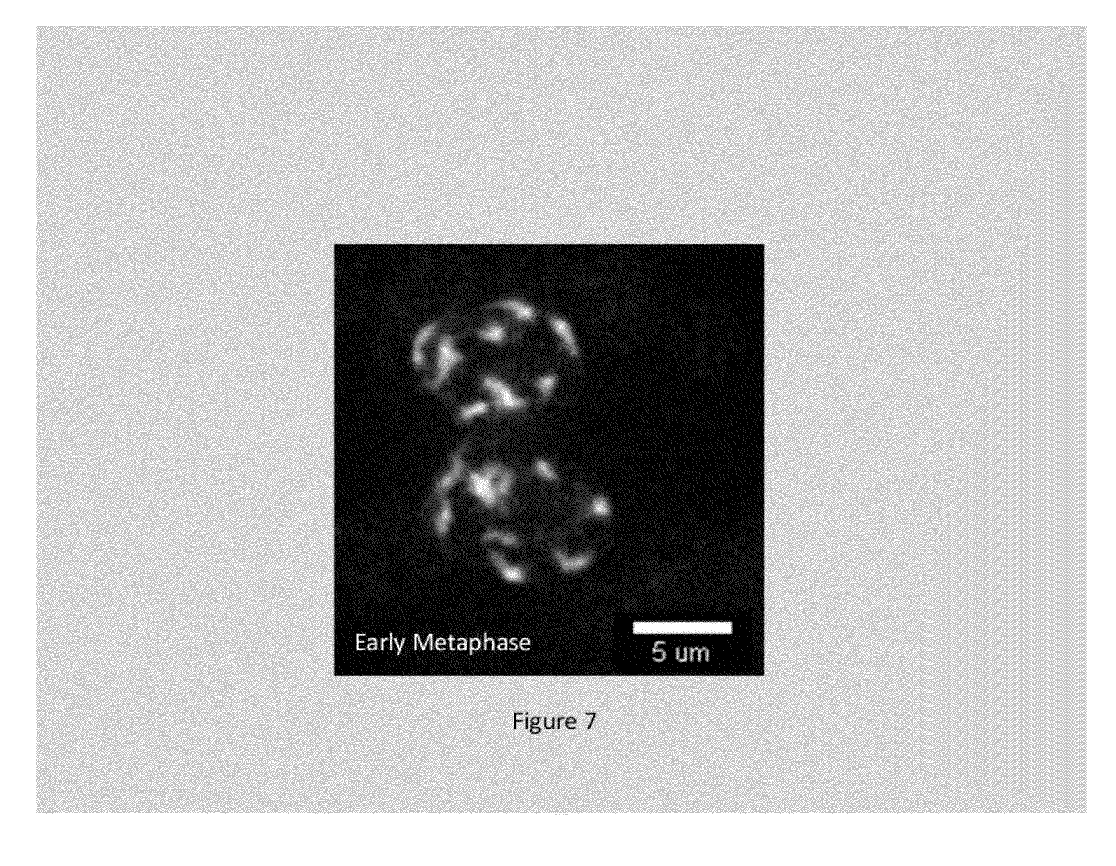

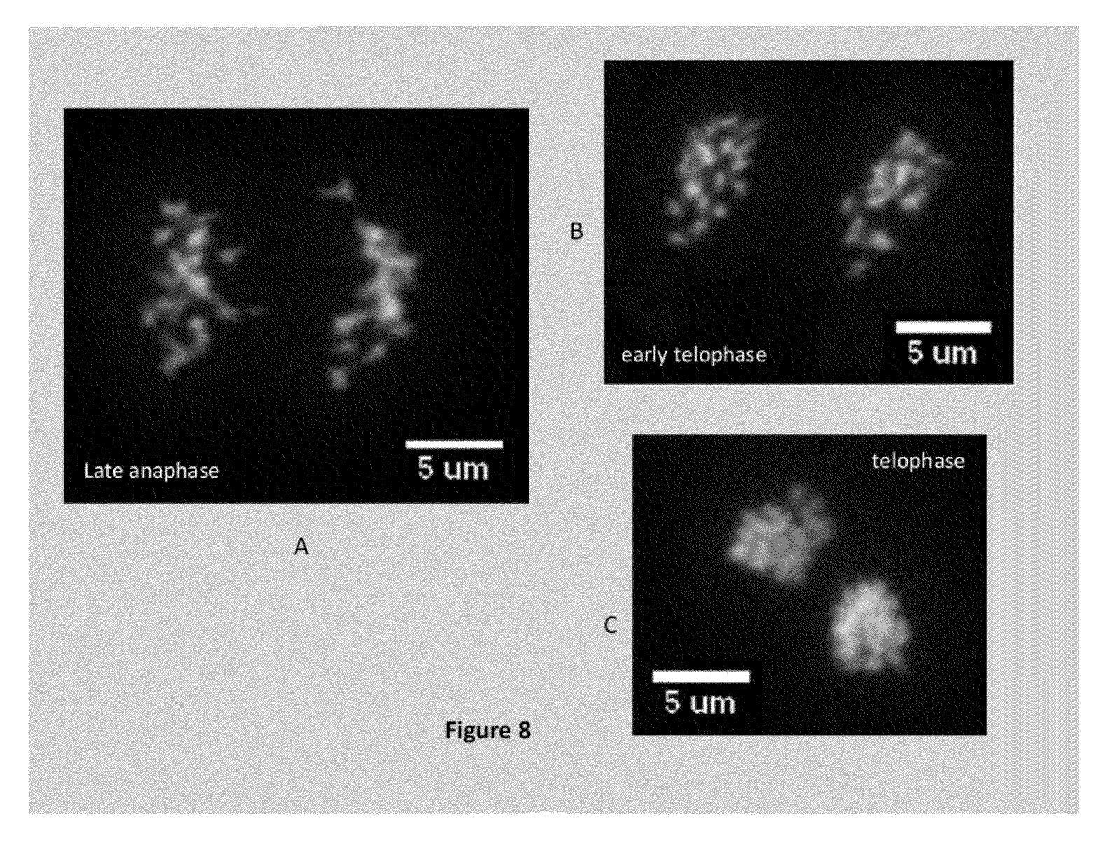

In 6A, T7 polymerase (for example) drives constitutive suppression of Meiosis genes leading to unreduced gametophytes. Then, Gal4DBD-VP16 (for example) drives suppression of CENH3 in the meiocytes, setting the stage for the CENH3 GFP-tailswap expression. Following that, an egg cell promoter drives expression of the CENH3 GFP-tailswap in the egg cell leading to female genome elimination in the first zygotic mitosis.

In 6B, AT-DD65 PRO and a Pollen PRO drives the WT CENH3 in the central cell and pollen allowing normal mitosis in the endosperm, and preventing genome elimination in the endosperm.

FIG. 7. Example of a mitotic spread of a php51198 T2 Arabidopsis seedling which does not display the MiMe phenotype. Two nuclei at early metaphase, each showing the diploid number of chromosomes (2n=10 chromosomes). Chomosomes are DAPI-Stained.

FIG. 8. Examples of mitotic spreads of MiMe T2 Arabidopsis seedlings showing the tetraploid number of chromosomes (4N=20) at various phases. Chomosomes are DAPI-Stained. 8A—Late anaphase, 8B—Early telophase, and 8C—telophase.

### DETAILED DESCRIPTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Apomixis

Apomixis, or asexual reproduction through seed, results in progeny that are genetic clones of the maternal parent. Apomixis requires a non-reduction of the chromosomes from one parental gamete and subsequent parthenogenic development of the embryo. Apomixis may provide a mechanism to maintain heterosis, or hybrid vigor, in crop plants. The present invention involves a combination of two technologies used to produce a self-reproducing hybrid. The first technology is a methodology to produce non-reduction of the genomic content of gametes or mitosis instead of meiosis (MiMe), as demonstrated in Arabidopsis (d′Erfurth, et al., (2009). PLoS Biol 7:e1000124). The second technology has the capacity to induce parent-specific genome elimination at high frequency (CENH3 GFP-tailswap) (Ravi and Chan, (2010) Nature 464:615-618). As used herein, “self-reproducing hybrid” refers to hybrid plants capable of perpetuating a heterozygous genome in progeny following self-fertilization. A demonstration of the capacity for these components to produce self-reproducing plants was shown by Marimuthu, et al., (2011) Science 331:876. However, the efficiency of this system is poor and requires significant modifications to become economically and biologically efficient. Specifically, the system demonstrated by Marimuthu, et al. (2011) does not provide for a self-perpetuating clonal line through a single plant line. It rather relies on a crossing of two distinct lines with each generation to perpetuate the clones, which would significantly limit the advantages of a stable hybrid production system. As disclosed here it is hypothesized that the demonstrated system by Marimuthu does not provide for consistent and reliable production of endosperm. Additionally, the genome elimination technology may disrupt some meiotic events, a potential cause of the aneuploidy observed in the system (Ravi, et al., (2011). Meiosis-Specific Loading of the Centromere-Specific Histone CENH3 in Arabidopsis thaliana. PLoS Genet. 7, e1002121. Epub 1002011 Jun3 1002129.). The methods described herein provide a means to overcome these limitations.

A. Mitosis Instead of Meiosis

Meiosis is a cell-division mechanism essential for sexually reproducing organisms. In plants, meiosis begins with one diploid cell containing two copies of each chromosome (2n) and produces four haploid gamete cells containing a single copy of each chromosome (1n). Traditional meiosis produces haploid gametes, each having a unique combination of maternal and paternal DNA. Meiosis typically involves chromosomal replication followed by recombination and two rounds of segregation and division. Alternatively, mitosis produces two identical daughter cells following a round of chromosomal replication, segregation, and division.

Inactivation of specific genes controlling meiosis can alter the chromosomal composition of the resultant gametes. For example, a mutation in the dyad gene of Arabidopsis resulted in female meiosis and megasporogenesis producing a dyad of megaspores, rather than a tetrad (Siddiqi, et al., (2000) Arabidopsis Development 127:197-207). By selectively inactivating a combination of meiosis-related genes, the second meiotic division can be replaced by a mitotic-like division, resulting in unreduced gametes that are identical to the parent cell (d′Erfurth, et al., (2009) PLoS Biol 7(6):e1000124). Inactivating osd1 resulted in an Arabidopsis mutant that did not undergo meiosis II, giving rise to diploid gametes having recombined chromosomes. Further, a double spo11-1/rec8 Arabidopsis mutant avoids the first division of meiosis and, instead, undergoes a mitotic-like division, followed by an unbalanced second division resulting in chromosomally unbalanced and sterile gametes. A triple osd1/spo11-1/rec8 mutant, designated MiMe, led to a mitotic-like first division due to the Atspo11-1 and Atrec8 mutations, and an absent second meiotic division due to the osd1 mutation. Thus, the MiMe mutation resulted in the replacement of meiosis with a mitotic-like division, thereby producing gametes having genetically identical chromosomes as the parent.

Various compositions are provided comprising suppression cassettes encoding inhibitory polynucleotides that decrease the activity of target polypeptides. In particular embodiments, silencing elements are provided encoding inhibitory polynucleotides that decrease the activity of Spo11-1, Rec8 or Osd1. In specific embodiments, silencing elements encoding inhibitory polynucleotides are provided that decrease the activity of Spo11-1, Rec8 and Osd1, thereby producing the MiMe phenotype. Such nucleic acid molecule constructs are referred to herein as “MiMe silencing elements”.

The Spo11 family of plant proteins are homologs of archaeal DNA topoisomerase VIA subunit (topo VIA), which participates in DNA replication. Spo11-1 specifically contributes to the creation of double stranded breaks necessary for recombination in the early phases of meiosis, and inactivating Spo11-1 results in sterile plants. Rec8 is responsible for localization of the axial chromosomal elements during meiosis. Following meiosis I, Rec8 has been identified at the centromere, and the depletion of Rec8 eliminated centromeric cohesion. Thus, the presence of Rec8 at the centromere has been thought to maintain sister chromatid cohesion throughout meiosis I (see, Nat Cell Biol 1:E125-7 (1999)). Osd1 (omission of second division) is an UVI4-like protein identified as a result of its co-regulation with other meiotic genes. In osd1 deficient Arabidopsis plants, the products of male meiosis were dyads instead of tetrads. Further, only tetrapoloid (4n) and triploid (3n) progeny were detected from self-pollinated osd1 deficient mutants. Thus, inactivation of osd1 produced functional diploid gametes due to absence of the second meiotic division.

In particular embodiments of the present invention, suppression cassettes provided elsewhere herein comprise MiMe silencing elements operably linked to promoters that drive expression in a plant. In some embodiments, promoters operably linked to MiMe silencing elements are inducible promoters. For example, in specific embodiments, MiMe silencing elements are operably linked to inducible promoters activated by a transactivator. As discussed elsewhere herein, the transactivator can be provided in the same plant or in a separate plant subsequently crossed with a plant comprising a MiMe silencing element operably linked to a transactivator-inducible promoter, thereby producing functional diploid gametes.

B. Genome Elimination

A method for producing plants that only inherit chromosomes from one parent can significantly accelerate plant breeding by providing plants in a single generation without the need for generations of inbreeding. By altering the structure of proteins of the kinetochore complex (centromere-specific polypeptides), such as CENH3, the chromosomes of the altered parent are eliminated in the zygote, thereby creating haploid plants. The resultant haploid plants have very high male sterility, but when pollinated by wild-type males, the female genome is eliminated at the first zygotic mitosis. In addition to near total male sterility, the resultant plants also show very low rates of female fecundity, likely due to female genome elimination in the endosperm. Egg cell-specific promoters can be useful in improving female fecundity associated with female genome elimination of the fertilized zygote by driving active CENH3 mutant expression in the egg cell. Egg cell-specific expression can maintain the female genome in the endosperm, thus ensuring the proper ratio of maternal to paternal chromosomes necessary for proper endosperm development. In some embodiments, active CENH3 mutant expression can be more widely expressed through the ovule, but a central cell promoter could be used to express a wild-type CENH3 thus “rescuing” the maternal genome in the resulting endosperm.

Various compositions that employ wild-type and modified kinetochore (centromere-specific) proteins are provided. Methods and compositions are provided comprising, for example, the CENH3, CENPC, MCM21, MIS12, NDC80 or NUF2 centromere-specific proteins. CENH3 proteins are discussed below. Structural and/or functional features of the other kinetochore proteins have been described in, for example, Du, et al., (2010) PLoS Genet. 6:e1000835; Talbert, et al., (2004) J. Biol. 3:18; Sato, et al., (2005) Chrom. Res. 13:827-834; Pidoux, et al., (2000) Opin. Cell Biol. 12:308-319; Du, et al., (2007) Chrom. Res. 15:767-775; Zhang and Dawe, (2011) Chrom. Res. (Mar. 19, 2011 epub) 1-10 and Meraldi, et al., (2006) Genome Biol. 7:R23, all of which are herein incorporated by reference.

In particular, various compositions that employ CENH3 and modified variants thereof are provided. CENH3 proteins are a well-characterized class of H3 histone protein variants associated with centromere function and development as one of the proteins that form the kinetochore complex. CENH3 proteins are characterized by a variable tail domain, which does not form a rigid secondary structure, and a conserved histone fold domain made up of three a-helical regions connected by loop sections. Additional structural and functional features of CENH3 proteins can be found in, e.g., Cooper, et al., (2004) Mol Biol Evol. 21(9):1712-8; Malik, et al., (2003) Nat Struct Biol. 10(11):882-91; Black, et al., (2008) Curr Opin Cell Biol. 20(1):91-100.

The CENH3 histone fold domain is conserved between CENH3 proteins from different species and can be distinguished by three a-helical regions connected by loop sections. While it will be appreciated that the exact location of the histone fold domain will vary in CENH3 variants, it will be found at the carboxyl terminus of an endogenous (wild-type) CENH3 protein. The border between the tail domain and the histone fold domain of CENH3 proteins is at, within, or near (i.e., within 5, 10, 15, 20 or 25 amino acids from the “P” of) the conserved PGTVAL (SEQ ID NO: 1) sequence. The PGTVAL sequence is approximately 81 amino acids from the N terminus of the Arabidopsis CENH3 protein, though the distance from the N terminus of different endogenous CENH3 proteins varies. Thus, in some embodiments, the histone fold region of CENH3 employed in the tailswap proteins includes all of the C-terminal amino acids of an endogenous CENH3 protein (or a protein substantially similar to the endogenous sequence) up to and including the PGTVAL. In other embodiments, the tailswap proteins can comprise more or less of the CENH3 sequence. For example, in some embodiments, the tailswap will comprise the C-terminal sequence of a CENH3 protein, but only up to an amino acid 5, 10, 15, 20 or 25 amino acids in the C-terminal direction from the “P” of the conserved PGTVAL sequence. In some embodiments, the tailswap will comprise the C-terminal sequence of a CENH3 protein, but only up to 5, 10, 15, 20 or 25 amino acids in the N-terminal direction from the “P” of the conserved PGTVAL sequence.

Any number of mutations of CENH3 can be introduced into a CENH3 protein to generate a mutated (including but not limited to a recombinantly altered) CENH3 protein capable of generating haploid plants when expressed in a plant having suppressed expression of an endogenous CENH3 protein and wherein wild-type CENH3 protein is provided to the resulting transgenic plant. For example, wild-type CENH3 can be provided by crossing a transgenic plant expressing an active CENH3 mutant to a plant expressing a wild-type CENH3 protein. Active CENH3 mutant proteins can be identified, for example, by random mutagenesis, by single or multiple amino acid targeted mutagenesis, by generation of complete or partial protein domain deletions, by fusion with heterologous amino acid sequences, or by combinations thereof. “Active centromere-specific mutant polypeptides refer to polypeptides that, when expressed in a plant in which the wild-type centromere-specific polypeptide is knocked out or inactivated, result in viable plants, which viable plants when crossed to a wild-type plant, produce haploid progeny at a more than normal frequency (e.g., at least 0.1, 0.5, 1, 5, 10, 20% or more). For example, “active CENH3 mutant proteins” refer to proteins that, when expressed in a plant in which CENH3 is knocked out or inactivated, result in viable plants, which viable plants when crossed to a wild-type plant, produce haploid progeny at a more than normal frequency (e.g., at least 0.1, 0.5, 1, 5, 10, 20% or more). Active mutated CENH3 proteins can be readily tested by recombinant expression of the mutated CENH3 protein in a plant lacking endogenous CENH3 protein, crossing the transgenic plant (as a male or female, depending on fertility) to a plant expressing wild-type CENH3 protein and then screening for the production of haploid progeny.

In some embodiments, an active CENH3 mutant protein is identical to an endogenous CENH3 protein but for 1, 2, 3, 4, 5, 6, 7, 8 or more (e.g., 1-2, 1-4, 1-8) amino acids. For example, in some embodiments, the endogenous wild-type protein from the plant is identical or substantially identical to SEQ ID NO: 5 and the active CENH3 mutant protein differs from the endogenous CENH3 protein by 1, 2, 3, 4, 5, 6, 7, 8 or more (e.g., 1-2, 1-4, 1-8) amino acids. It is believed that active CENH3 mutants include, for example, proteins comprising: a heterologous amino acid sequence (including but not limited to green fluorescent protein (GFP)) linked to a CENH3 truncated or complete tail domain or non-CENH3 tail domain, either of which is linked to a CENH3 histone fold domain or a CENH3 truncated tail domain, the heterologous CENH3 tail domain or non-CENH3 tail domain, either of which is linked to a CENH3 histone fold domain. In some embodiments, the active CENH3 mutant protein comprises a fusion of an amino-terminal heterologous amino acid sequence to the histone-fold domain of a CENH3 protein. Generally, the histone fold domain will be identical or at least substantially identical to the CENH3 protein endogenous to the organism in which the active CENH3 mutant protein will be expressed. In some embodiments, the active CENH3 mutant protein will include a histone tail domain, which can be, for example, a non-CENH3 tail domain, or a CENH3 tail domain.

It is believed that a large number of different amino acid sequences, when linked to a protein comprising a CENH3 histone-fold domain and a sequence that can function as or replace a histone tail domain, can be used to construct an active CENH3 mutant. In some embodiments, a heterologous sequence is linked directly to the CENH3 histone-fold domain.

In some embodiments, the heterologous sequence is an intervening amino acid sequence linked to the CENH3 histone-fold domain. In some embodiments, the intervening amino acid sequence is an intact or truncated CENH3 tail domain. The heterologous amino acid sequence, in combination with the histone-fold domain, will be sufficient to prevent the lethality associated with loss of endogenous CENH3, but will sufficiently disrupt centromeres to allow for production of haploid progeny, as discussed herein. Thus, in some embodiments, the heterologous amino acid sequence will comprise a portion that is, or mimics the function of, a histone tail domain and optionally can also comprise a bulky amino acid sequence that disrupts centromere function. In certain embodiments, at least a portion of the heterologous amino acid sequence of the mutated CENH3 protein comprises any amino acid sequence of at least 10, 20, 30, 40, 50, e.g., 10-30, 10-50, 20-50, 30-50 amino acids, optionally lacking a stable secondary structure (e.g., lacking coils, helices or beta-sheets). In some embodiments, the tail domain has less than 90, 80 or 70% identity with the tail domain (e.g., the N-terminal 135 amino acids) of the CENH3 protein endogenous to the organism in which the mutated CENH3 protein will be expressed. In some embodiments, the tail domain of the mutated CENH3 protein comprises the tail domain of a non-CENH3 histone protein, including but not limited to an H3 histone protein. In some embodiments, the tail domain of the mutated CENH3 protein comprises the tail domain of a non-CENH3 histone protein endogenous to the organism in which the mutated CENH3 protein will be expressed. In some embodiments, the tail domain of the mutated CENH3 protein comprises the tail domain of a homologous or orthologous (from a different plant species) CENH3 tail. For example, it has been found that GFP fused to a maize CENH3 tail domain linked to an Arabidopsis CENH3 histone-fold domain is active.

As noted above, in some embodiments, the tail domain of an H3 histone (not to be confused with a CENH3 histone) is used as the tail domain portion of the active CENH3 mutant protein (these embodiments are sometimes referred to as “tailswap” proteins). Plant H3 tail domains are well conserved in various organisms.

In some embodiments, active CENH3 mutant proteins will lack at least a portion (e.g., at least 5, 10, 15, 20, 25, 30 or more amino acids) of the endogenous CENH3 N-terminal region, and thus, in some embodiments, will have a truncated CENH3 tail domain compared to a wild-type endogenous CENH3 protein. Active CENH3 mutant proteins may, or may not, be linked to a heterologous sequence.

Optionally, the heterologous amino acid sequence can comprise, or further comprise, one or more amino acid sequences at the amino and/or carboxyl terminus and/or linking the tail and histone fold domains. For example, in some embodiments, the active CENH3 mutant protein (e.g., a tailswap or other active CENH3 mutant protein) comprises a heterologous amino acid sequence linked to the amino end of the tail domain. In some embodiments, the heterologous sequence is linked to the amino terminus of an otherwise wild-type CENH3 protein, wherein the heterologous sequence interferes with centromere function. For example, it has been found that GFP, when linked to wild-type CENH3, sufficiently disrupts centromeres to allow for production of haploid progeny. It is believed that the heterologous sequence can be any sequence that disrupts the CENH3 protein's ability to maintain centromere function. Thus, in some embodiments, the heterologous sequence comprises an amino acid sequence of at least 5, 10, 15, 20, 25, 30, 50 or more kD.

In some embodiments, the active CENH3 mutant protein will comprise a protein domain that acts as a detectable or selectable marker. For example, an exemplary selectable marker protein is fluorescent or an antibiotic or herbicide resistance gene product. Selectable or detectable protein domains are useful for monitoring the presence or absence of the mutated CENH3 protein in an organism.

In other embodiments, expression cassettes are provided comprising an active CENH3 mutant protein operably linked to a promoter that drives expression in a plant. In particular embodiments, promoters operably linked to active CENH3 mutant proteins are inducible promoters or tissue-specific promoters. For example, in specific embodiments, active CENH3 mutant proteins are operably linked to promoters specifically induced in the ovule of a plant.

In some embodiments, expression cassettes comprising a nucleotide sequence encoding wild-type CENH3 operably linked to a promoter that drives expression in a plant are provided. In particular embodiments, promoters operably linked to nucleotide sequences encoding wild-type CENH3 are tissue specific promoters. For example, nucleotide sequences encoding wild-type CENH3 operably linked to central cell-specific promoters (e.g., AT-DD65 promoter) that drive expression of wild-type CENH3 in the central cell of a plant are provided. Expression cassettes comprising a central-cell specific promoter operably linked to a polynucleotide encoding wild-type CENH3 can be provided in the same parental plant as CENH3 suppression cassettes and/or the same parental plant as active CENH3 mutant expression cassettes.

Further provided are inhibitory polynucleotides that decrease the activity of wild-type CENH3. In some embodiments, suppression cassettes comprising a silencing element encoding inhibitory polynucleotides that decrease the activity of wild-type CENH3 operably linked to an inducible promoter that drives expression in a plant are provided. In specific embodiments, suppression cassettes comprising a silencing element encoding inhibitory polynucleotides that decrease the activity of wild-type CENH3 operably linked to a promoter specifically induced by a transactivator are provided. As discussed elsewhere herein, the transactivator can be provided in the same plant or in a separate plant subsequently crossed with a plant comprising a CENH3 silencing element operably linked to a transactivator-inducible promoter, thereby activating the CENH3 silencing element in the progeny plant. In some embodiments, a recombinase may be used to eliminate a buffering component between a promoter and the DNA region encoding the inhibitory polynucleotides.

In a particular embodiment, a first plant comprising a CENH3 expression cassette comprising a central cell-specific promoter, a CENH3 suppression cassette comprising a transactivator A-inducible promoter and a transactivator B expression cassette comprising an ovule-specific promoter is crossed with a second plant comprising an active CENH3 mutant expression cassette comprising an ovule-specific promoter, a MiMe suppression cassette comprising a transactivator B-inducible promoter and a transactivator A expression cassette comprising an ovule-specific promoter, producing a tetraploid zygote that subsequently loses the female genome from the egg cell following a generation of self fertilization, ultimately resulting in a self-reproducing hybrid progeny plant.

C. Methods for Producing Self-Reproducing Hybrid Plants

A single-cross hybrid plant results from the cross of two inbred varieties, each of which has a genotype that complements the genotype of the other. A hybrid progeny of the first generation is designated F1. In the development of commercial hybrids in a plant breeding program, the F1 hybrid plants are most desired. F1 hybrids are more vigorous than their inbred parents. This hybrid vigor, or heterosis, can be manifested in many polygenic traits, including increased vegetative growth and increased yield.

Crossing a pollen parent plant comprising cassettes for suppressing the activity of an endogenous kinetochore complex protein (e.g., CENH3, CENPC, MCM21, MIS12, NDC80 or NUF2 protein) in progeny ovules and cassettes for expressing an endogenous kinetochore complex protein in the central cell of progeny to an ovule parent plant comprising cassettes for expressing inhibitory polynucleotides resulting in a MiMe phenotype in progeny and cassettes for expressing an active mutated kinetochore complex protein (e.g., a tailswap or other mutated CENH3 or non-CENH3 kinetochore complex protein) in the ovule of progeny as described herein, will result in at least some progeny (e.g., at least 0.1%, 0.5%, 1%, 5%, 10%, 20% or more) that are diploid following self-fertilization and comprise only chromosomes from the male parent that expresses the kinetochore complex protein. Thus, the present invention allows for the generation of diploid plants capable of self-reproducing.

While the present invention is not known to depend on a particular mechanism, it is believed that the methods of the present invention increase self-reproducing hybrid seed viability by preventing female genome elimination in the central cell of the ovule. It is further believed that complementing the central cell with wild-type CENH3 allows proper endosperm development by maintaining a 2M:1P (2 maternal:1 paternal) ratio necessary for proper endosperm development.

In some embodiments, a method for producing a self-reproducing hybrid plant is provided comprising crossing a first plant comprising a first suppression cassette comprising a MiMe silencing element and a first expression cassette expressing an active CENH3 mutant protein with a second plant comprising a second suppression cassette that reduces the level of wild-type CENH3 and a second expression cassette expressing CENH3 specifically in the central cell. Self fertilization of the resultant progeny plant results in the elimination of the female diploid genome in the zygote and normal development of the endosperm, thereby producing a self-reproducing hybrid plant.

### II. Compositions

Compositions disclosed herein provide nucleic acid molecule constructs comprising expression and suppression cassettes comprising polynucleotides related to meiosis or genome elimination. As used herein, “meiosis-related” or “MiMe-related” refers to those polynucleotides encoding polypeptides involved directly or indirectly in the process of meiosis. Further, as used herein, “kinetochore” or “CENH3” refers to the specialized protein structure on chromosomes that mediates the attachment of spindle fibers during cell division.

Decreasing the level of polynucleotides encoding such polypeptides or decreasing the activity of the encoded polypeptides could result in absence of the first meiotic division, meiosis II, or unbalanced second meiotic divisions. Methods for measuring the level of polynucleotides and activity of the encoded polypeptides are disclosed elsewhere herein. For example, RNA transcripts are monitored through the use of qRT-PCR. SybrGreen or TaqMan probes may be used. Polypeptide activities are assayed indirectly through cytogenetics and progeny segregation analysis.

By “reduces”, “reducing”, “decrease” or “decreasing” the expression level of a polynucleotide or activity of a polypeptide encoded thereby is intended to mean, the polynucleotide or polypeptide level of the target sequence is statistically lower than the polynucleotide level or polypeptide level of the same target sequence in an appropriate control plant that is not expressing the silencing element. In particular embodiments of the invention, reducing the polynucleotide level and/or the polypeptide level of the target sequence in a plant according to the invention results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control plant. Methods to assay for the level of the RNA transcript, the level of the encoded polypeptide or the activity of the polynucleotide or polypeptide are known in the art and discussed elsewhere herein.

TABLE 1

| | | | POLYNUCLEOTIDE/ |
|---|---|---|---|
| | | | POLYPEPTIDE |
| SEQ ID. | ORGANISM NAME | DESCRIPTION | (PN/PP) |
| SEQ ID NO: 1 | ARTIFICIAL SEQUENCE | CONSERVED | PP |
| | | DOMAIN | |
| SEQ ID NO: 2 | ARABIDOPSIS THALIANA | SPO11-1 | PN |
| SEQ ID NO: 3 | ARABIDOPSIS THALIANA | 3ISD | PN |
| SEQ ID NO: 4 | ARABIDOPSIS THALIANA | REC8 | PN |
| SEQ ID NO: 5 | ARABIDOPSIS THALIANA | CENH3 | PN |
| SEQ ID NO: 6 | ARTIFICIAL SEQUENCE | PRIMER | PN |
| SEQ ID NO: 7 | ARTIFICIAL SEQUENCE | PRIMER | PN |
| SEQ ID NO: 8 | ARTIFICIAL SEQUENCE | PRIMER | PN |
| SEQ ID NO: 9 | ARTIFICIAL SEQUENCE | PRIMER | PN |
| SEQ ID NO: 10 | ARTIFICIAL SEQUENCE | PRIMER | PN |
| SEQ ID NO: 11 | ARTIFICIAL SEQUENCE | PRIMER | PN |
| SEQ ID NO: 12 | ARTIFICIAL SEQUENCE | PRIMER | PN |
| SEQ ID NO: 13 | BRASSICA NAPUS | CENH3 | PN |
| SEQ ID NO: 14 | BRASSICA RAPA | CENH3 | PN |
| SEQ ID NO: 15 | BRASSICA RAPA | CENH3 | PP |
| SEQ ID NO: 16 | GLYCINE MAX | CENH3 | PN |
| SEQ ID NO: 17 | GLYCINE MAX | CENH3 | PN |
| SEQ ID NO: 18 | MEDICAGO TRUNCATULA | CENH3 | PN |
| SEQ ID NO: 19 | MEDICAGO TRUNCATULA | CENH3 | PP |
| SEQ ID NO: 20 | ORYZA SATIVA | CENH3 | PN |
| SEQ ID NO: 21 | ORYZA SATIVA | CENH3 | PP |
| SEQ ID NO: 22 | ORYZA SATIVA | CENH3 | PN |
| SEQ ID NO: 23 | ORYZA SATIVA | CENH3 | PP |
| SEQ ID NO: 24 | SETARIA ITALICA | CENH3 | PN |
| SEQ ID NO: 25 | SETARIA ITALICA | CENH3 | PP |
| SEQ ID NO: 26 | SORGHUM BICOLOR | CENH3 | PN |
| SEQ ID NO: 27 | SORGHUM BICOLOR | CENH3 | PP |
| SEQ ID NO: 28 | VITIS VINIFERA | CENH3 | PN |
| SEQ ID NO: 29 | VITIS VINIFERA | CENH3 | PP |
| SEQ ID NO: 30 | ZEA MAYS | CENH3 | PN |
| SEQ ID NO: 31 | ZEA MAYS | CENH3 | PP |
| SEQ ID NO: 32 | BRASSICA NAPUS | OSD1 | PN |
| SEQ ID NO: 33 | BRASSICA RAPA | OSD1 | PN |
| SEQ ID NO: 34 | BRASSICA RAPA | OSD1 | PP |
| SEQ ID NO: 35 | BRASSICA RAPA | OSD1 | PN |
| SEQ ID NO: 36 | BRASSICA RAPA | OSD1 | PP |
| SEQ ID NO: 37 | GLYCINE MAX | OSD1 | PN |
| SEQ ID NO: 38 | GLYCINE MAX | OSD1 | PP |
| SEQ ID NO: 39 | GLYCINE MAX | OSD1 | PN |
| SEQ ID NO: 40 | GLYCINE MAX | OSD1 | PP |
| SEQ ID NO: 41 | MEDICAGO TRUNCATULA | OSD1 | PN |
| SEQ ID NO: 42 | MEDICAGO TRUNCATULA | OSD1 | PP |
| SEQ ID NO: 43 | ORYZA SATIVA | OSD1 | PN |
| SEQ ID NO: 44 | ORYZA SATIVA | OSD1 | PP |
| SEQ ID NO: 45 | ORYZA SATIVA | OSD1 | PN |
| SEQ ID NO: 46 | ORYZA SATIVA | OSD1 | PP |
| SEQ ID NO: 47 | SORGHUM BICOLOR | OSD1 | PN |
| SEQ ID NO: 48 | SORGHUM BICOLOR | OSD1 | PP |
| SEQ ID NO: 49 | VITIS VINIFERA | OSD1 | PN |
| SEQ ID NO: 50 | VITIS VINIFERA | OSD1 | PP |
| SEQ ID NO: 51 | ZEA MAYS | OSD1 | PN |
| SEQ ID NO: 52 | ZEA MAYS | OSD1 | PP |
| SEQ ID NO: 53 | BRASSICA NAPUS | SPO11-1 | PN |
| SEQ ID NO: 54 | BRASSICA NAPUS | SPO11-1 | PP |
| SEQ ID NO: 55 | BRASSICA RAPA | SPO11-1 | PN |
| SEQ ID NO: 56 | BRASSICA RAPA | SPO11-1 | PP |
| SEQ ID NO: 57 | GLYCINE MAX | SPO11-1 | PN |
| SEQ ID NO: 58 | GLYCINE MAX | SPO11-1 | PP |
| SEQ ID NO: 59 | GLYCINE MAX | SPO11-1 | PN |
| SEQ ID NO: 60 | GLYCINE MAX | SPO11-1 | PP |
| SEQ ID NO: 61 | GLYCINE MAX | SPO11-1 | PN |
| SEQ ID NO: 62 | GLYCINE MAX | SPO11-1 | PP |
| SEQ ID NO: 63 | MEDICAGO TRUNCATULA | SPO11-1 | PN |
| SEQ ID NO: 64 | MEDICAGO TRUNCATULA | SPO11-1 | PP |
| SEQ ID NO: 65 | ORYZA SATIVA | SPO11-1 | PN |
| SEQ ID NO: 66 | ORYZA SATIVA | SPO11-1 | PP |
| SEQ ID NO: 67 | SETARIA ITALICA | SPO11-1 | PN |
| SEQ ID NO: 68 | SETARIA ITALICA | SPO11-1 | PP |
| SEQ ID NO: 69 | SORGHUM BICOLOR | SPO11-1 | PN |
| SEQ ID NO: 70 | SORGHUM BICOLOR | SPO11-1 | PP |
| SEQ ID NO: 71 | VITIS VINIFERA | SPO11-1 | PN |
| SEQ ID NO: 72 | VITIS VINIFERA | SPO11-1 | PP |
| SEQ ID NO: 73 | ZEA MAYS | SPO11-1 | PN |
| SEQ ID NO: 74 | ZEA MAYS | SPO11-1 | PP |
| SEQ ID NO: 75 | BRASSICA NAPUS | REC8 | PN |
| SEQ ID NO: 76 | BRASSICA RAPA | REC8 | PN |
| SEQ ID NO: 77 | BRASSICA RAPA | REC8 | PP |
| SEQ ID NO: 78 | GLYCINE MAX | REC8 | PN |
| SEQ ID NO: 79 | GLYCINE MAX | REC8 | PP |
| SEQ ID NO: 80 | GLYCINE MAX | REC8 | PN |
| SEQ ID NO: 81 | GLYCINE MAX | REC8 | PP |
| SEQ ID NO: 82 | MEDICAGO TRUNCATULA | REC8 | PN |
| SEQ ID NO: 83 | MEDICAGO TRUNCATULA | REC8 | PP |
| SEQ ID NO: 84 | MEDICAGO TRUNCATULA | REC8 | PN |
| SEQ ID NO: 85 | MEDICAGO TRUNCATULA | REC8 | PP |
| SEQ ID NO: 86 | ORYZA SATIVA | REC8 | PN |
| SEQ ID NO: 87 | ORYZA SATIVA | REC8 | PP |
| SEQ ID NO: 88 | SETARIA ITALICA | REC8 | PN |
| SEQ ID NO: 89 | SETARIA ITALICA | REC8 | PP |
| SEQ ID NO: 90 | SORGHUM BICOLOR | REC8 | PN |
| SEQ ID NO: 91 | SORGHUM BICOLOR | REC8 | PP |
| SEQ ID NO: 92 | VITIS VINIFERA | REC8 | PN |
| SEQ ID NO: 93 | VITIS VINIFERA | REC8 | PP |
| SEQ ID NO: 94 | ZEA MAYS | REC8 | PN |
| SEQ ID NO: 95 | ZEA MAYS | REC8 | PP |
| SEQ ID NO: 96 | BRASSICA NAPUS | CENP-C | PN |
| SEQ ID NO: 97 | BRASSICA NAPUS | CENP-C | PP |
| SEQ ID NO: 98 | BRASSICA NAPUS | CENP-C | PN |
| SEQ ID NO: 99 | BRASSICA NAPUS | CENP-C | PP |
| SEQ ID NO: 100 | BRASSICA RAPA | CENP-C | PN |
| SEQ ID NO: 101 | BRASSICA RAPA | CENP-C | PP |
| SEQ ID NO: 102 | BRASSICA RAPA | CENP-C | PN |
| SEQ ID NO: 103 | GLYCINE MAX | CENP-C | PN |
| SEQ ID NO: 104 | GLYCINE MAX | CENP-C | PP |
| SEQ ID NO: 105 | GLYCINE MAX | CENP-C | PN |
| SEQ ID NO: 106 | GLYCINE MAX | CENP-C | PP |
| SEQ ID NO: 107 | MEDICAGO TRUNCATULA | CENP-C | PN |
| SEQ ID NO: 108 | MEDICAGO TRUNCATULA | CENP-C | PP |
| SEQ ID NO: 109 | ORYZA SATIVA | CENP-C | PN |
| SEQ ID NO: 110 | ORYZA SATIVA | CENP-C | PP |
| SEQ ID NO: 111 | SETARIA ITALICA | CENP-C | PN |
| SEQ ID NO: 112 | SETARIA ITALICA | CENP-C | PP |
| SEQ ID NO: 113 | SORGHUM BICOLOR | CENP-C | PN |
| SEQ ID NO: 114 | SORGHUM BICOLOR | CENP-C | PP |
| SEQ ID NO: 115 | ZEA MAYS | CENP-C | PN |
| SEQ ID NO: 116 | ZEA MAYS | CENP-C | PP |
| SEQ ID NO: 117 | ZEA MAYS | CENP-C | PN |
| SEQ ID NO: 118 | ZEA MAYS | CENP-C | PP |
| SEQ ID NO: 119 | ZEA MAYS | CENP-C | PN |
| SEQ ID NO: 120 | ZEA MAYS | CENP-C | PP |
| SEQ ID NO: 121 | BRASSICA NAPUS | MIS12 | PN |
| SEQ ID NO: 122 | BRASSICA NAPUS | MIS12 | PN |
| SEQ ID NO: 123 | BRASSICA NAPUS | MIS12 | PP |
| SEQ ID NO: 124 | BRASSICA RAPA | MIS12 | PN |
| SEQ ID NO: 125 | BRASSICA RAPA | MIS12 | PP |
| SEQ ID NO: 126 | GLYCINE MAX | MIS12 | PN |
| SEQ ID NO: 127 | GLYCINE MAX | MIS12 | PP |
| SEQ ID NO: 128 | GLYCINE MA | MIS12 | PN |
| SEQ ID NO: 129 | GLYCINE MAX | MIS12 | PP |
| SEQ ID NO: 130 | MEDICAGO TRUNCATULA | MIS12 | PN |
| SEQ ID NO: 131 | MEDICAGO TRUNCATULA | MIS12 | PP |
| SEQ ID NO: 132 | MEDICAGO TRUNCATULA | MIS12 | PN |
| SEQ ID NO: 133 | MEDICAGO TRUNCATULA | MIS12 | PP |
| SEQ ID NO: 134 | ORYZA SATIVA | MIS12 | PN |
| SEQ ID NO: 135 | ORYZA SATIVA | MIS12 | PP |
| SEQ ID NO: 136 | SORGHUM BICOLOR | MIS12 | PN |
| SEQ ID NO: 137 | SORGHUM BICOLOR | MIS12 | PP |
| SEQ ID NO: 138 | VITIS VINIFERA | MIS12 | PN |
| SEQ ID NO: 139 | VITIS VINIFERA | MIS12 | PP |
| SEQ ID NO: 140 | ZEA MAYS | MIS12 | PN |
| SEQ ID NO: 141 | ZEA MAYS | MIS12 | PP |
| SEQ ID NO: 142 | ZEA MAYS | MIS12 | PN |
| SEQ ID NO: 143 | ZEA MAYS | MIS12 | PP |
| SEQ ID NO: 144 | BRASSICA NAPUS | NUF2 | PN |
| SEQ ID NO: 145 | BRASSICA NAPUS | NUF2 | PP |
| SEQ ID NO: 146 | BRASSOCA NAPUS | NUF2 | PN |
| SEQ ID NO: 147 | BRASSICA RAPA | NUF2 | PN |
| SEQ ID NO: 148 | BRASSICA RAPA | NUF2 | PP |
| SEQ ID NO: 149 | GLYCINE MAX | NUF2 | PN |
| SEQ ID NO: 150 | GLYCINE MAX | NUF2 | PP |
| SEQ ID NO: 151 | MEDICAGO TRUNCATULA | NUF2 | PN |
| SEQ ID NO: 152 | MEDICAGO TRUNCATULA | NUF2 | PP |
| SEQ ID NO: 153 | MEDICAGO TRUNCATULA | NUF2 | PN |
| SEQ ID NO: 154 | MEDICAGO TRUNCATULA | NUF2 | PP |
| SEQ ID NO: 155 | ORYZA SATIVA | NUF2 | PN |
| SEQ ID NO: 156 | ORYZA SATIVA | NUF2 | PP |
| SEQ ID NO: 157 | ORYZA SATIVA | NUF2 | PN |
| SEQ ID NO: 158 | ORYZA SATIVA | NUF2 | PP |
| SEQ ID NO: 159 | SETARIA ITALICA | NUF2 | PN |
| SEQ ID NO: 160 | SETARIA ITALICA | NUF2 | PP |
| SEQ ID NO: 161 | SORGHUM BICOLOR | NUF2 | PN |
| SEQ ID NO: 162 | SORGHUM BICOLOR | NUF2 | PP |
| SEQ ID NO: 163 | SORGHUM BICOLOR | NUF2 | PN |
| SEQ ID NO: 164 | SORGHUM BICOLOR | NUF2 | PP |
| SEQ ID NO: 165 | SORGHUM BICOLOR | NUF2 | PN |
| SEQ ID NO: 166 | SORGHUM BICOLOR | NUF2 | PP |
| SEQ ID NO: 167 | VITIS VINIFERA | NUF2 | PN |
| SEQ ID NO: 168 | VITIS VINIFERA | NUF2 | PP |
| SEQ ID NO: 169 | VITIS VINIFERA | NUF2 | PN |
| SEQ ID NO: 170 | VITIS VINIFERA | NUF2 | PP |
| SEQ ID NO: 171 | ZEA MAYS | NUF2 | PN |
| SEQ ID NO: 172 | ZEA MAYS | NUF2 | PP |
| SEQ ID NO: 173 | ZEA MAYS | NUF2 | PN |
| SEQ ID NO: 174 | ZEA MAYS | NUF2 | PP |
| SEQ ID NO: 175 | BRASSICA NAPUS | PRD1 | PN |
| SEQ ID NO: 176 | BRASSICA RAPA | PRD1 | PN |
| SEQ ID NO: 177 | BRASSICA RAPA | PRD1 | PP |
| SEQ ID NO: 178 | GLYCINE MAX | PRD1 | PN |
| SEQ ID NO: 179 | GLYCINE MAX | PRD1 | PP |
| SEQ ID NO: 180 | GLYCINE MAX | PRD1 | PN |
| SEQ ID NO: 181 | GLYCINE MAX | PRD1 | PP |
| SEQ ID NO: 182 | MEDICAGO TRUNCATULA | PRD1 | PN |
| SEQ ID NO: 183 | MEDICAGO TRUNCATULA | PRD1 | PP |
| SEQ ID NO: 184 | ORYZA SATIVA | PRD1 | PN |
| SEQ ID NO: 185 | ORYZA SATIVA | PRD1 | PP |
| SEQ ID NO: 186 | SETARIA ITALICA | PRD1 | PN |
| SEQ ID NO: 187 | SETARIA ITALICA | PRD1 | PP |
| SEQ ID NO: 188 | SORGHUM BICOLOR | PRD1 | PN |
| SEQ ID NO: 189 | SORGHUM BICOLOR | PRD1 | PP |
| SEQ ID NO: 190 | VITIS VINIFERA | PRD1 | PN |
| SEQ ID NO: 191 | VITIS VINIFERA | PRD1 | PP |
| SEQ ID NO: 192 | ZEA MAYS | PRD1 | PN |
| SEQ ID NO: 193 | ZEA MAYS | PRD1 | PP |
| SEQ ID NO: 194 | BRASSICA NAPUS | PRD2 | PN |
| SEQ ID NO: 195 | BRASSICA RAPA | PRD2 | PN |
| SEQ ID NO: 196 | BRASSICA RAPA | PRD2 | PP |
| SEQ ID NO: 197 | BRASSICA RAPA | PRD2 | PN |
| SEQ ID NO: 198 | BRASSICA RAPA | PRD2 | PP |
| SEQ ID NO: 199 | GLYCINE MAX | PRD2 | PN |
| SEQ ID NO: 200 | GLYCINE MAX | PRD2 | PP |
| SEQ ID NO: 201 | GLYCINE MAX | PRD2 | PN |
| SEQ ID NO: 202 | GLYCINE MAX | PRD2 | PP |
| SEQ ID NO: 203 | MEDICAGO TRUNCATULA | PRD2 | PN |
| SEQ ID NO: 204 | MEDICAGO TRUNCATULA | PRD2 | PP |
| SEQ ID NO: 205 | ORYZA SATIVA | PRD2 | PN |
| SEQ ID NO: 206 | ORYZA SATIVA | PRD2 | PP |
| SEQ ID NO: 207 | SETARIA ITALICA | PRD2 | PN |
| SEQ ID NO: 208 | SETARIA ITALICA | PRD2 | PP |
| SEQ ID NO: 209 | SORGHUM BICOLOR | PRD2 | PN |
| SEQ ID NO: 210 | SORGHUM BICOLOR | PRD2 | PP |
| SEQ ID NO: 211 | VITIS VINIFERA | PRD2 | PN |
| SEQ ID NO: 212 | VITIS VINIFERA | PRD2 | PP |
| SEQ ID NO: 213 | ZEA MAYS | PRD2 | PN |
| SEQ ID NO: 214 | :ZEA MAYS | PRD2 | PP |
| SEQ ID NO: 215 | BRASSICA NAPUS | PRD3 | PN |
| SEQ ID NO: 216 | BRASSICA RAPA | PRD3 | PN |
| SEQ ID NO: 217 | BRASSICA RAPA | PRD3 | PP |
| SEQ ID NO: 218 | BRASSICA RAPA | PRD3 | PN |
| SEQ ID NO: 219 | BRASSICA RAPA | PRD3 | PP |
| SEQ ID NO: 220 | GLYCINE MAX | PRD3 | PN |
| SEQ ID NO: 221 | GLYCINE MAX | PRD3 | PP |
| SEQ ID NO: 222 | GLYCINE MAX | PRD3 | PN |
| SEQ ID NO: 223 | GLYCINE MAX | PRD3 | PP |
| SEQ ID NO: 224 | MEDICAGO TRUNCATULA | PRD3 | PN |
| SEQ ID NO: 225 | MEDICAGO TRUNCATULA | PRD3 | PP |
| SEQ ID NO: 226 | ORYZA SATIVA | PRD3 | PN |
| SEQ ID NO: 227 | ORYZA SATIVA | PRD3 | PP |
| SEQ ID NO: 228 | SETARIA ITALICA | PRD3 | PN |
| SEQ ID NO: 229 | SETARIA ITALICA | PRD3 | PP |
| SEQ ID NO: 230 | SORGHUM BICOLOR | PRD3 | PN |
| SEQ ID NO: 231 | SORGHUM BICOLOR | PRD3 | PP |
| SEQ ID NO: 232 | VITIS VINIFERA | PRD3 | PN |
| SEQ ID NO: 233 | VITIS VINIFERA | PRD3 | PP |
| SEQ ID NO: 234 | VITIS VINIFERA | PRD3 | PN |
| SEQ ID NO: 235 | ZEA MAYS | PRD3 | PN |
| SEQ ID NO: 236 | ZEA MAYS | PRD3 | PP |
| SEQ ID NO: 237 | ZEA MAYS | PRD3 | PN |
| SEQ ID NO: 238 | ZEA MAYS | PRD3 | PP |
| SEQ ID NO: 239 | ARABIDOPSIS THALIANA | CENP-O | PN |
| SEQ ID NO: 240 | ARABIDOPSIS THALIANA | CENP-O | PP |
| SEQ ID NO: 241 | BRASSICA NAPUS | CENP-O | PN |
| SEQ ID NO: 242 | BRASSICA RAPA | CENP-O | PN |
| SEQ ID NO: 243 | BRASSICA RAPA | CENP-O | PP |
| SEQ ID NO: 244 | GLYCINE MAX | CENP-O | PN |
| SEQ ID NO: 245 | GLYCINE MAX | CENP-O | PP |
| SEQ ID NO: 246 | GLYCINE MAX | CENP-O | PN |
| SEQ ID NO: 247 | GLYCINE MAX | CENP-O | PP |
| SEQ ID NO: 248 | GLYCINE MAX | CENP-O | PN |
| SEQ ID NO: 249 | GLYCINE MAX | CENP-O | PP |
| SEQ ID NO: 250 | MEDICAGO TRUNCATULA | CENP-O | PN |
| SEQ ID NO: 251 | MEDICAGO TRUNCATULA | CENP-O | PP |
| SEQ ID NO: 252 | ORYZA SATIVA | CENP-O | PN |
| SEQ ID NO: 253 | ORYZA SATIVA | CENP-O | PP |
| SEQ ID NO: 254 | SETARIA ITALICA | CENP-O | PN |
| SEQ ID NO: 255 | SETARIA ITALICA | CENP-O | PP |
| SEQ ID NO: 256 | SETARIA ITALICA | CENP-O | PN |
| SEQ ID NO: 257 | SETARIA ITALICA | CENP-O | PP |
| SEQ ID NO: 258 | SETARIA ITALICA | CENP-O | PN |
| SEQ ID NO: 259 | SETARIA ITALICA | CENP-O | PP |
| SEQ ID NO: 260 | SORGHUM BICOLOR | CENP-O | PN |
| SEQ ID NO: 261 | SORGHUM BICOLOR | CENP-O | PP |
| SEQ ID NO: 262 | SORGHUM BICOLOR | CENP-O | PN |
| SEQ ID NO: 263 | SORGHUM BICOLOR | CENP-O | PN |
| SEQ ID NO: 264 | VITIS VINIFERA | CENP-O | PN |
| SEQ ID NO: 265 | VITIS VINIFERA | CENP-O | PP |
| SEQ ID NO: 266 | ZEA MAYS | CENP-O | PN |
| SEQ ID NO: 267 | ZEA MAYS | CENP-O | PP |
| SEQ ID NO: 268 | ZEA MAYS | CENP-O | PN |
| SEQ ID NO: 269 | ZEA MAYS | CENP-O | PP |
| SEQ ID NO: 270 | ZEA MAYS | CENP-O | PN |
| SEQ ID NO: 271 | ZEA MAYS | CENP-O | PP |
| SEQ ID NO: 272 | GLYCINE MAX | REC8 | PN |
| SEQ ID NO: 273 | GLYCINE MAX | REC8 | PN |
| SEQ ID NO: 274 | GLYCINE MAX | REC8 | PN |

A. Silencing Elements

Further provided are nucleic acid molecules comprising nucleotide sequences encoding inhibitory nucleic acids, and fragments and variants thereof that are useful in decreasing the level of proteins responsible for normal meiosis and wild-type kinetochore activity. Such fragments and variants are useful in silencing elements and suppression cassettes.

By “silencing elements” is intended polynucleotides that can reduce or eliminate the expression level of a target sequence by influencing the level of the target RNA transcript or, alternatively, by influencing translation and thereby affecting the level of the encoded polypeptide. As used herein, a “target sequence” or “target polynucleotide” comprises any sequence that one desires to reduce the level of expression. In specific embodiments, the target sequence comprises the nucleotide sequence set forth in SEQ ID NO: 2, 3 and 4 and decreasing the level of expression of the target sequence results in an alteration of normal meiosis activity. In other embodiments, the target sequence comprises the nucleotide sequence set forth in SEQ ID NO: 5. Methods to assay for functional silencing elements that are capable of reducing or eliminating the level of a sequence of interest are known in the art.

As discussed in further detail below, silencing elements can include, but are not limited to, a sense suppression element, an antisense suppression element, a double stranded RNA, an siRNA, an amiRNA, an miRNA or a hairpin suppression element. Non-limiting examples of silencing elements that can be employed to decreased expression of meiosis-related genes or CENH3 genes comprise fragments and variants of the sense or antisense sequence of the sequences set forth in SEQ ID NOs: 2, 3, 4 and/or 5. In other embodiments, dominant negative mutants or protein fragments may be used to suppress target function.

i. Sense Suppression Elements

Silencing elements of the invention may comprise a sense suppression element. As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323, herein incorporated by reference. The sense suppression element can be any length so long as it allows for the suppression of the targeted sequence. The sense suppression element can be, for example, the full-length nucleotide sequence of SEQ ID NO: 2, 3, 4 and 5 or about 10, 15, 16, 17, 18, 19, 20, 22, 25, 30, 50, 100, 150, 200, 250, 300, 350, 400, 450, 500 nucleotides or longer of the nucleotides set forth in SEQ ID NO: 2, 3, 4 and 5. In other embodiments, the sense suppression element can be, for example, the full-length nucleotide sequence of SEQ ID NO: 2, 3, 4 and 5 or about 10, 15, 16, 17, 18, 19, 20, 22, 25, 30, 50, 100, 150, 200, 250, 300, 350, 400, 450, 500, 600, 700, 900, 1000, 1100, 1200, 1300, 1400, 1500 nucleotides or longer of the nucleotides set forth in SEQ ID NO: 2, 3, 4 and 5.

ii. Antisense Suppression Elements

Silencing elements of the invention may comprise an antisense suppression element. As used herein, an “antisense suppression element” comprises a polynucleotide that is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98% or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide.

In one example, sequences of at least about 15, 16, 17, 18, 19, 20, 22, 25, 50, 100, 200, 300, 400, 450, 500 nucleotides or longer of the nucleotides set forth in SEQ ID NO: 2, 3, 4 and 5 or a complement thereof, may be used. In another example, sequences of at least about 15, 16, 17, 18, 19, 20, 22, 25, 50, 100, 200, 300, 400, 450, 500, 600, 700, 900, 1000, 1100, 1200, 1300, 1400, 1500 nucleotides or longer of the nucleotides set forth in SEQ ID NO: 2, 3, 4 and 5 or a complement thereof, may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference.

iii. Double Stranded RNA Suppression Element

Silencing elements of the invention may comprise a double stranded RNA silencing element. A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions of the invention mediate the reduction of expression of a target sequence, for example, by mediating RNA interference “RNAi” or gene silencing in a sequence-specific manner. In the context of the present invention, the dsRNA is capable of reducing or eliminating the level or expression of a target polynucleotide or the polypeptide encoded thereby in a plant.

The dsRNA can reduce or eliminate the expression level of the target sequence by influencing the level of the target RNA transcript, by influencing translation and thereby affecting the level of the encoded polypeptide or by influencing expression at the pre-transcriptional level (i.e., via the modulation of chromatin structure, methylation pattern, etc., to alter gene expression). See, for example, Verdel, et al., (2004) Science 303:672-676; Pal-Bhadra, et al., (2004) Science 303:669-672; Allshire, (2002) Science 297:1818-1819; Volpe, et al., (2002) Science 297:1833-1837; Jenuwein, (2002) Science 297:2215-2218 and Hall, et al., (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (sRNA), double-stranded RNA (dsRNA), micro-RNA (miRNA), artificial micro-RNA (amiRNA), hairpin RNA, short hairpin RNA (shRNA), post-transcriptional gene silencing RNA (ptgsRNA) and others.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double-stranded structure. Multiple structures can be employed as hairpin elements. In specific embodiments, the dsRNA suppression element comprises a hairpin element that comprises in the following order, a first segment, a second segment and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication Number WO 2002/00904, herein incorporated by reference. In specific embodiments, the loop region can be optimized to be as short as possible while still providing enough intramolecular flexibility to allow the formation of the base-paired stem region. Accordingly, the loop sequence is generally less than about 1500, 1400, 1300, 1200, 1100, 1000, 900, 800, 700, 600, 500, 400, 300, 200, 100, 50, 25, 20, 19, 18, 17, 16, 15, 10 nucleotides or less. The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

In specific embodiments, the sequences used in the first, the second and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000 or more than 1000 nucleotides that share sufficient sequence identity to the target polynucleotide to allow for a decrease in expression levels of the target polynucleotide when expressed in an appropriate cell.

In further embodiments, the domain of the first, the second and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99% or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hairpin RNA constructs to silence gene expression in vivo has been described by Panstruga, et al., (2003) Mol. Biol. Rep. 30:135-140, herein incorporated by reference.

The amount of complementarity shared between the first, second and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s) and any combination thereof. In some instances, to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers, et al., (2003) J. Biol. Chem 278:7108-7118 and Yang, et al., (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

In particular embodiments, the hairpin RNAs of the invention may also comprise an intron. For such intron-containing hairpin RNAs, the interfering molecules have the same general structure as for the hairpin RNAs described herein above, but the RNA molecule additionally comprises an intron that is capable of being spliced in the cell in which the hairpin RNA is expressed. The use of an intron minimizes the size of the loop in the hairpin RNA molecule following splicing and this increases the efficiency of interference. See, for example, Smith, et al., (2000) Nature 407:319-320. In fact, Smith, et al., show 100% suppression of endogenous gene expression using intron-containing hairpin RNA-mediated interference. Methods for using intron-containing hairpin RNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz, et al., (2002) PNAS 99(4):16499-16506 and Mette, et al., (2000) EMBO J 19(19):5194-5201.

In other embodiments, the dsRNA can comprise a small RNA (sRNA). sRNAs can comprise both micro RNA (miRNA) and short-interfering RNA (siRNA) (Meister and Tuschl, (2004) Nature 431:343-349 and Bonetta, et al., (2004) Nature Methods 1:79-86). miRNAs are regulatory agents comprising about 19 ribonucleotides which are highly efficient at inhibiting the expression of target polynucleotides. See, for example, Javier, et al., (2003) Nature 425:257-263, herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

When expressing an miRNA, it is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto, et al., (2004) Genes & Development 18:2237-2242 and Guo, et al., (2005) Plant Cell 17:1376-1386).

Artificial microRNAs (amiRNAs) have recently been described in Arabidopsis targeting viral mRNA sequences (Niu, et al., (2006) Nature Biotechnology 24:1420-1428) or endogenous genes (Schwab, et al., (2006) Plant Cell 18:1121-1133). The amiRNA construct can be expressed under different promoters in order to change the spatial pattern of silencing (Schwab, et al., (2006) Plant Cell 18:1121-1133). Artificial miRNAs replace the microRNA and its complementary star sequence in a precursor miRNA and substitute sequences that target an mRNA to be silenced. Silencing by endogenous miRNAs can be found in a variety of spatial, temporal and developmental expression patterns (Parizotto, et al., (2007) Genes Dev 18:2237-2242; Alvarez, et al., (2006) Plant Cell 18:1134-51). Artificial miRNA can be constructed to both capture and extend the diversity and specificity in the patterns of silencing.

The methods and compositions of the invention can employ silencing elements that, when transcribed, form a dsRNA molecule. Accordingly, the heterologous polynucleotide being expressed need not form the dsRNA by itself, but can interact with other sequences in the plant cell to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, US Patent Application Publication 2007/0130653, entitled “Methods and Compositions for Gene Silencing”, herein incorporated by reference. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

In specific embodiments, the compositions of the invention include nucleic acid molecules that comprise the nucleotide sequence of Spo11-1 (SEQ ID NO: 2), Osd1 (SEQ ID NO: 3), Rec8 (SEQ ID NO: 4) and CENH3 (SEQ ID NO: 5) nucleotide sequences. Alternatively, such nucleic acid molecules comprise a nucleotide sequence that selectively hybridizes with SEQ ID NOS: 2, 3, 4 and/or 5. Furthermore, such isolated polynucleotides may comprise a nucleotide sequence comprising the complementary sequence to SEQ ID NOS: 2, 3, 4 and/or 5 or the complementary sequence to a nucleotide sequence that selectively hybridizes with SEQ ID NOS: 2, 3, 4 and/or 5.

B. Transactivator Elements

Transactivator elements are provided herein for use in regulating the expression of genes of interest by selectively activating inducible promoters. For example, the polynucleotides encoding transactivator proteins of the invention can be placed under the control of a constitutive, tissue-specific or other transactivator-inducible promoter to control the expression of a nucleotide of interest operably linked to a transactivator-inducible promoter. In some embodiments, a polynucleotide encoding a transactivator protein can be provided on an expression cassette in a separate plant from the expression or suppression cassette comprising the corresponding transactivator-inducible promoter. Expression cassettes provided herein comprising polynucleotides encoding transactivator proteins can further comprise operably linked promoters that drive expression of the transactivator in a plant. As used herein, “transactivator A” and “transactivator B” refer to any transactivator element used for regulating the expression of genes of interest by selectively activating inducible promoters. Examples of transactivators include the GAL4DBD-VP16/UAS PRO system, the T7 polymerase/T7 PRO system and the LexA transactivator system commonly known in the art or any combination thereof (Yagi, et al., (2010) Proc. Natl. Acad. Sci. 107(37):16166-16171).

As used herein, “transactivator promoter” refers to a promoter operably linked to a polynucleotide encoding a transactivator. In specific embodiments, expression cassettes are provided encoding a polynucleotide encoding a transactivator operably linked to a constitutive or tissue-specific promoter. For example, the tissue-specific promoter operably linked to a polynucleotide encoding a transactivator can be an ovule-specific promoter wherein the transactivator is specifically expressed in the ovule of a plant. Such a transactivator specifically expressed in the ovule of a plant can activate the corresponding transactivator-inducible promoter resulting in the expression of a gene of interest only in the ovule. In one embodiment of the invention, a first plant comprising an expression cassette comprising a polynucleotide encoding transactivator A operably linked to an ovule-specific promoter is crossed with a second plant comprising a suppression cassette comprising a CENH3 silencing element operably linked to a transactivator A-inducible promoter. In the resulting progeny plant, the CENH3 silencing element is specifically expressed in the ovule.

In another embodiment of the invention, a first plant comprising an expression cassette comprising a polynucleotide encoding transactivator B under the control of a constitutive promoter is crossed with a second plant comprising a suppression cassette comprising a MiMe silencing element under the control of a transactivator-inducible promoter. In progeny from the resulting cross, the transactivator activates constitutive expression of the MiMe silencing element. In certain embodiments, an expression cassette comprising a polynucleotide encoding transactivator A is provided in the same plant as a suppression cassette comprising a transactivator B-inducible promoter, wherein transactivator A does not activate the expression of the transactivator B-inducible promoter.

C. Expression Cassettes and Suppression Cassettes

Compositions of the invention also encompass expression cassettes and suppression cassettes. It is recognized that the polynucleotides and silencing elements of the invention can be provided in expression cassettes and suppression cassettes, respectively, for expression in a plant of interest. Expression cassettes provided herein may comprise, for example, polynucleotides encoding a transactivator, an active CENH3 mutant and/or wild-type CENH3 or fragments or variants thereof. Suppression cassettes provided herein may, for example, comprise a silencing element as described herein above.

The expression and suppression cassettes of the invention can include 5′ and 3′ regulatory sequences operably linked to the polynucleotide or silencing elements of the invention. “Operably linked” is intended to mean a functional linkage between two or more elements. For example, an operable linkage between a polynucleotide and a regulatory sequence (i.e., a promoter) is a functional link that allows for expression of the polynucleotide of the invention. In particular examples, a polynucleotide or silencing element of the invention can be operably linked to a promoter that drives expression in a plant. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression and suppression cassettes can be provided with a plurality of restriction sites and/or recombination sites for insertion of the polynucleotide to be under the transcriptional regulation of the regulatory regions. The expression and suppression cassettes may additionally contain selectable marker genes.

The expression and suppression cassettes can include in the 5′-3′ direction of transcription, a transcriptional and translational initiation region (i.e., a promoter), a polynucleotide encoding a polypeptide or the silencing element(s) employed in the methods and compositions of the invention and a transcriptional and translational termination region (i.e., termination region) functional in plants. In those embodiments, where the suppression cassettes encode double stranded RNA the suppression cassette can comprise two convergent promoters that drive transcription of the operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA.

The regulatory regions (i.e., promoters, transcriptional regulatory regions, and translational termination regions) and/or the polynucleotides or silencing elements employed in the invention may be native/analogous to the host cell or to each other. Alternatively, the regulatory regions and/or the polynucleotides or silencing elements employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding a polypeptide or silencing element, may be native with the plant host or may be derived from another source (i.e., foreign or heterologous) to the promoter, the polynucleotide, the silencing element, the plant host or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression or suppression cassettes of the invention, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In particular embodiments, the silencing element of a suppression cassette may be operably linked to a promoter that drives expression of the silencing element in a plant. In other embodiments, polynucleotides encoding an active CENH3 mutant, wild-type CENH3 or transactivator of an expression cassette may be operably linked to a promoter that drives expression of the polynucleotide in a plant. It is recognized that a number of promoters can be used in the practice of the invention. Polynucleotides encoding silencing elements can be combined with constitutive, tissue-preferred, transactivator-inducible or other promoters for expression in plants.

Such constitutive promoters include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

An inducible promoter, for instance, a transactivator-inducible promoter are provided. For example, transactivator-inducible promoters for use in the expression or suppression cassettes disclosed herein include: Gal4DBD::VP16/UAS; Gal4DBD::hypothetical activator domain/UAS; T7 Polymerase/T7 promoter; other proprietary systems; in theory: unique DNA binding domain::activation domain/DNA recognition element::minimal promoter element as demonstrated in numerous novel fusions in plant transient experimental systems.

Chemical-regulated promoters can be used to modulate the expression of a gene in a plant through the application of an exogenous chemical regulator. Depending upon the objective, the promoter may be a chemical-inducible promoter, where application of the chemical induces gene expression or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Egg cell-specific promoters, central cell-specific promoters, and pollen specific promoters can be utilized to confine expression of silencing elements, active CENH3 mutants or wild-type CENH3 to the egg, central cell, or pollen of a plant. For example, AT-DD45 PRO, AT-RKD1 PRO or AT-RKD2 PRO can be used as egg cell-specific promoters. The egg and central cell-specific MEA (FIS1) and FIS2 promoters are also useful reproductive tissue-specific promoters (Luo, et al., (2000) Proc. Natl. Acad. Sci. USA 97:10637-10642; Vielle-Calzada, et al., (1999) Genes Dev. 13:2971-2982). Other examples of egg cell and central cell-specific promoters can be found, for example, in Steffen, et al., (2007) Plant J 51:281-292 and Ohnishi, et al., (2011) Plant Physiology 155: 881-891, herein incorporated by reference in their entirety. For example, central cell specific promoters from Steffen, et al., can be used, including, for example, AT-DD7 PRO, AT-DD9 PRO, AT-DD22 PRO, AT-DD25 PRO, AT-DD36 PRO, AT-DD41 PRO, AT-DD66 PRO and AT-DD65 PRO.

Ovule-specific promoters are known and can be selected for ovule-specific expression of polynucleotides disclosed elsewhere herein. For example, ovule-specific promoters can drive expression of transactivators or active CENH3 mutants in the entire ovule, including, but not limited to the egg cell and central cell. The ovule-specific promoter for BEL1 gene can also be used (Reiser, et al., (1995) Cell 83:735-742 GenBank Number U39944; Ray, et al., (1994) Proc. Natl. Acad. Sci. USA 91:5761-5765) as well as those disclosed in U.S. patent application Ser. No. 12/912,231, filed Oct. 26, 2010 herein incorporated by reference in its entirety.

Possible promoters also include the Black Cherry promoter for Prunasin Hydrolase (PH DL1.4 PRO) (U.S. Pat. No. 6,797, 859), Thioredoxin H promoter from cucumber and rice (Fukuda, et al., (2005). Plant Cell Physiol. 46(11):1779-86), Rice (RSs1) (Shi, et al., (1994). J. Exp. Bot. 45(274):623-631) and maize sucrose synthese -1 promoters (Yang, et al., (1990) PNAS 87:4144-4148), PP2 promoter from pumpkin Guo, et al., (2004) Transgenic Research 13:559-566), At SUC2 promoter (Truernit, et al., (1995) Planta 196(3):564-70., At SAM-1 (S-adenosylmethionine synthetase) (Mijnsbrugge, et al., (1996) Planr. Cell. Physiol. 37(8): 1108-1115) and the Rice tungro bacilliform virus (RTBV) promoter (Bhattacharyya-Pakrasi, et al., (1993) Plant J. 4(1):71-79).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Sci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

D. Fragments and Variants

The expression and suppression cassettes of the invention can be designed based on the naturally occurring CENH3, Spo11-1, Rec8 or Osd1 polynucleotides or fragments or variants thereof. By “fragment” is intended a portion of the nucleotide sequence. Fragments of the disclosed nucleotide sequences may range from at least about 10, 16, 20, 50, 75, 100, 150, 200, 250, 300, 350, 400, 450 or 500 contiguous nucleotides, or up to the number of nucleotides present in a full-length CENH3, Spo11-1, Rec8 or Osd1 polynucleotide disclosed herein (for example, 1089 nucleotides for SEQ ID NO: 2) so long as the fragment achieves the desired objective, i.e., expression of a biologically active polypeptide of interest (for example, the active CENH3 mutant or CENH3 polypeptide) or expression of a functional silencing element that suppresses expression or function of the CENH3, Spo11-1, Rec8 or Osd1 polypeptide.

By “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide comprises a naturally occurring nucleotide sequence, for example, a naturally occurring CENH3, Spo11-1, Rec8 or Osd1 polynucleotide. For polynucleotides, naturally occurring variants can be identified with the use of well-known molecular biology techniques such as, for example, polymerase chain reaction (PCR) and hybridization techniques as outlined elsewhere herein. Variant polynucleotides also include synthetically derived polynucleotides, such as those generated, for example, by using site-directed mutagenesis. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters commonly known in the art.

In particular embodiments, a silencing element of the invention may comprise the full-length nucleotide sequence of SEQ ID NOS: 2, 3, 4 and/or 5 or a fragment of the nucleotide sequence of SEQ ID NOS: 2, 3, 4 and/or 5. Additionally, silencing elements of the invention may comprise a variant of the full-length nucleotide sequence of SEQ ID NOS: 2, 3, 4 and/or 5 or a variant of a fragment of the nucleotide sequence of SEQ ID NOS: 2, 3, 4 and/or 5. Such variants will maintain at least 80% sequence identity to the nucleotide sequence of the native full-length sequence or fragment from which the variant is derived. It is recognized that the CENH3 and active CENH3 mutants can be altered in various ways including amino acid substitutions, deletions, truncations and insertions. Methods for such manipulations are generally known in the art. Nucleotide sequence variants and fragments of the CENH3, Spo11-1, Rec8 or Osd1 gene can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

Thus, the expression and suppression cassettes can be based on the naturally occurring nucleotide sequences as well as variations and modified forms thereof. Such variants will continue to possess the desired activity. Obviously, where a functional polypeptide is to be expressed, the mutations that will be made in the DNA encoding the variant polypeptide must not place the sequence out of reading frame and optimally will not create complementary regions that could produce secondary mRNA structure. See, EP Patent Application Publication Number 75,444.

The deletions, insertions and substitutions of the encoded polypeptides encompassed herein are not expected to produce radical changes in the characteristics of the protein. However, when it is difficult to predict the exact effect of the substitution, deletion or insertion in advance of doing so, one skilled in the art will appreciate that the effect will be evaluated by routine screening assays. Deletions, insertions and substitutions within a polynucleotide of interest are made such that the variant polynucleotide retains the desired activity, i.e., encoding a functional CENH3 variant or encoding a functional silencing element that effectively suppresses expression or function of the CENH3, Spo11-1, Rec8 or Osd1 polypeptide. In an inbred situation, analyses of protein functionality would be best done through cytogenetic evaluations, i.e., microscopy of meiotic stages and resultant products. Mis-function of these proteins would have impacts on fertility and offspring health (across reasonable numbers of plants) which would be in most cases readily noticed. In crosses between differing genetic backgrounds, molecular markers could be used to assess recombination and segregation.

### III. Plants

Plants, plant cells, plant parts and seeds and grain comprising one or more of the expression cassettes and suppression cassettes described elsewhere herein are provided. In specific embodiments, the plants and/or plant parts comprise stably incorporated in the genome at least one transactivator expression cassette, at least one active CENH3 mutant expression cassette, at least one wild-type CENH3 expression cassette, at least one MiMe suppression cassette and/or at least one wild-type CENH3 suppression cassette. Thus, the invention provides plants, plant cells, plant parts and seed that have stably incorporated into their genome a transactivator A expression cassette, an active CENH3 mutant expression cassette and a MiMe suppression cassette. Further provided are plants, plant cells, plant parts and seeds that have stably incorporated into their genome a transactivator B expression cassette, a wild-type CENH3 expression cassette, and a wild-type CENH3 suppression cassette. In specific embodiments, progeny plants are provided resulting from the cross of a plant having stably incorporated into the genome a transactivator A expression cassette, an active CENH3 mutant expression cassette and a MiMe suppression cassette with a plant having stably incorporated into the genome a transactivator B expression cassette, a wild-type CENH3 expression cassette and a wild-type CENH3 suppression cassette wherein the progeny plant is a self-reproducing hybrid plant. Such self-reproducing hybrid progeny plants comprise at least one transactivator expression cassette, at least one active CENH3 mutant expression cassette, at least one wild-type CENH3 expression cassette, at least one MiMe suppression cassette and/or at least one wild-type CENH3 suppression cassette.

In specific embodiments, plants and seeds are provided comprising a suppression cassette comprising a MiMe silencing element operably linked to a transactivator B-inducible promoter, an expression cassette comprising a polynucleotide encoding an active CENH3 mutant operably linked to an ovule-specific promoter, and an expression cassette comprising a polynucleotide encoding a transactivator A operably linked to an ovule-specific promoter. In other embodiments, plants and seeds are provided comprising a suppression cassette comprising a wild-type CENH3 silencing element operably linked to a transactivator A-inducible promoter, an expression cassette comprising a polynucleotide encoding a wild-type CENH3 polypeptide operably linked to a central-cell specific promoter and an expression cassette comprising a polynucleotide encoding a transactivator B operably linked to a promoter.

As used herein, the term plant includes plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the invention, provided that these parts comprise the introduced polynucleotides.

The expression cassettes and suppression cassettes disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffee spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis) and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.). In other embodiments corn and soybean plants are optimal and in yet other embodiments soybean plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In some embodiments, the polynucleotides comprising the expression cassettes or suppression cassettes described elsewhere herein are engineered into a molecular stack. Thus, the various plants, plant cells and seeds disclosed herein can further comprise one or more traits of interest, and in more specific embodiments, the plant, plant part or plant cell is stacked with any combination of polynucleotide sequences of interest, expression cassettes of interest or suppression cassettes of interest in order to create plants with a desired combination of traits. As used herein, the term “stacked” includes having the multiple traits present in the same plant.

These stacked combinations can be created by any method including, but not limited to, breeding plants by any conventional methodology or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

Thus, in specific embodiments, the expression cassettes and suppression cassettes disclosed herein function to produce self-reproducing hybrid progeny plants when combined in a progeny plant. Such expression and suppression cassettes can then be stacked with any other sequence of interest, including polynucleotides conferring herbicide tolerance. Non-limiting examples of such sequences are disclosed elsewhere herein.

A “subject plant or plant cell” is one in which genetic alteration, such as transformation, has been affected as to a polynucleotide of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The methods of the invention comprise introducing expression and suppression cassettes disclosed herein into the genome of a plant or plant cell. The methods provided herein do not depend on a particular method for introducing polynucleotides comprising the expression or suppression cassettes into the host cell, only that the polynucleotide gains access to the interior of at least one cell of the host. Methods for introducing polynucleotides into host cells (i.e., plants) are known in the art and include, but are not limited to, stable transformation methods, transient transformation methods and virus-mediated methods.

“Stable transformation” is intended to mean that the nucleotide construct introduced into a host (i.e., a plant) integrates into the genome of the plant and is capable of being inherited by the progeny thereof. “Transient transformation” is intended to mean that a polynucleotide is introduced into the host (i.e., a plant) and expressed temporally.

Transformation protocols as well as protocols for introducing polynucleotide sequences into plants may vary depending on the type of plant or plant cell, i.e., monocot or dicot, targeted for transformation. Suitable methods of introducing polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (Townsend, et al., U.S. Pat. No. 5,563,055; Zhao, et al., U/S/ Pat. No. 5,981,840), direct gene transfer (Paszkowski, et al., (1984) EMBO J. 3:2717-2722) and ballistic particle acceleration (see, for example, Sanford, et al., U.S. Pat. No. 4,945,050; Tomes, et al., U.S. Pat. No. 5,879,918; Tomes, et al., U.S. Pat. No. 5,886,244; Bidney, et al., U.S. Pat. No. 5,932,782; Tomes, et al., (1995) “Direct DNA Transfer into Intact Plant Cells via Microprojectile Bombardment,” in Plant Cell, Tissue, and Organ Culture: Fundamental Methods, ed. Gamborg and Phillips, (Springer-Verlag, Berlin); McCabe, et al., (1988) Biotechnology 6:923-926) and Led transformation (WO 2000/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Tomes, U.S. Pat. No. 5,240,855; Buising, et al., U.S. Pat. Nos. 5,322,783 and 5,324,646; Tomes, et al., (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; Bowen, et al., U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D′Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

In specific embodiments, the expression and suppression cassettes disclosed herein can be provided to a plant using a variety of transient transformation methods. Such transient transformation methods include, but are not limited to, the introduction of the expression and suppression cassettes directly into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91: 2176-2180 and Hush, et al., (1994) The Journal of Cell Science 107:775-784, all of which are herein incorporated by reference.

Alternatively, expression and suppression cassettes can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, expression and suppression cassettes disclosed herein may be introduced into plants by contacting plants with a virus or viral nucleic acids. Generally, such methods involve incorporating a nucleotide construct of the invention within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the invention can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown and either pollinated with the same transformed strain or different strains and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having expression and suppression cassettes disclosed herein, stably incorporated into their genome.

### EXAMPLES

### Example 1

### Plant Material and Growth Conditions

Plants were grown in artificial soil mix at 20° C. under fluorescent lighting. Wild-type and mutant strains of Arabidopsis were obtained from ABRC, Ohio or NASC, UK. dyad was crossed to the No-0 strain to generate populations that were heterozygous for markers across the genome. MiMe plants were a mixture of Col-0 from Atspo11-1-3/Atrec8-3 and No-0 from osd1-1 (S1). The GEM plants used in this study are F1 progeny obtained by crossing cenh3-1/cenh3-1 GFP-tailswap/GFP-tailswap (female) to cenh3-1/cenh3-1 GFPCENH3/GFP-CENH3 (male).

cenh3-1 was isolated by the TILLING procedure (Comai and Henikoff, (2006) Plant J 45:684-94). The TILLING population was created by mutagenizing Arabidopsis thaliana in the Col-0 accession with ethylmethanesulfonate, using standard protocols. Cenh3-1 was isolated by TILLING using the CEL1 heteroduplex cleavage assay, with PCR primers specific for the CENH3/HTR12 gene. Cloning of the GFP-CENH3 and GFP-tailswap transgenes and construction of the complemented cenh3-1 GFP-CENH3 and cenh3-1 GFP-tailswap lines are described elsewhere herein (Ravi, et al, manuscript in preparation).

To cross wild-type as the female to GFP-tailswap as the male, a dissecting microscope was used to directly observe pollen deposition on the stigma (GFP-tailswap is mostly male-sterile). The amount of viable pollen in individual flowers of GFP-tailswap varies. Flowers that clearly showed higher amounts of pollen were selected and pollinated with more than 60 anthers (10 GFP-tailswap flowers) per wild-type stigma to achieve the seed set reported in Table 1. Using an optivisor (magnifying lens) and approximately 12 anthers (2 GFP-tailswap flowers) per wild-type stigma, a much lower seed set per silique was obtained. Seed from GFP-tailswap X wild-type crosses were sown on 1 X MS plates containing 1% sucrose to maximize germination efficiency, particularly of seed that had an abnormal appearance. Late germinating seeds were frequently haploid.

A chimera was created in which the A. thaliana CENH3 tail from CENH3 is replaced with the CENH3 tail domain from maize (Zea mays), thereby generating a fusion of the maize CENH3 tail and A. thaliana CENH3 histone-fold domain and transformed the fusion into cenh3-1 heterozygotes. As expected, this GFP-maize tailswap protein was targeted to kinetochores and rescued the embryo-lethal phenotype of cenh3-1.

### Example 2

### Genotyping and Microsatellite Marker Analysis

Primers for osd1-1, Atspo11-1-3 and Atrec8-3 (MiMe) genotyping are described (S1). Microsatellite markers were analyzed. Primer sequences were obtained from TAIR (www.Arabidopsis.org) or from the MSAT database (INRA). cenh3-1: a point mutation G161A in the CENH3 gene (also known as, HTR12) detected with dCAPS primers (dCAPs restriction polymorphism with EcoRV, the wild-type allele cuts):

| (SEQ ID NO: 6) | |
|---|---|
| Primer 1: | GGTGCGATTTCTCCAGCAGTAAAAATC |
| (SEQ ID NO: 7) | |
| Primer 2: | CTGAGAAGATGAAGCACCGGCGATAT |

Detection of GFP-tailswap insertion on chromosome 1:

| Primer 1 for wild-type and T-DNA: | |
|---|---|
| CACATACTCGCTACTGGTCAGAGAATC | (SEQ ID NO: 8) |
| Primer 2 for wild-type only: | |
| CTGAAGCTGAACCTTCGTCTCG | (SEQ ID NO: 9) |
| Primer 3 for the T-DNA: | |
| AATCCAGATCCCCCGAATTA | (SEQ ID NO: 10) |

Primers for detection of GFP-CENH3:

| | |
|---|---|
| CAGCAGAACACCCCCATC | (SEQ ID NO: 11) |
| (in GFP) | |
| CTGAGAAGATGAAGCACCGGCGATAT | (SEQ ID NO: 12) |
| (in CENH3) | |

### Ploidy Analysis

MiMe and osd1 offspring ploidy analyses were performed by flow cytometry and systemically confirmed by chromosome spreads. For dyad offspring, ploidy analysis was by flow cytometry and randomly selected diploid eliminants (n=5) were further confirmed by FISH analysis using a centromere repeat probe to count chromosomes and all were found to be diploids. Isolation of nuclei for flow cytometry was performed. Flow cytometry analysis was carried out using an internal diploid and tetraploid control to unambiguously identify diploid plants.

In elimination crosses to the wild-type tetraploid line (C24 background), triploids were identified as late flowering (due to combination of the Col-0 FRIGIDA and C24 FLOWERING LOCUS C alleles). The aneuploid plants show distinct morphological phenotypes such as altered vegetative growth, variation in rosette leaf morphology (size and shape), a range of leaf color (pale yellow to dark green) and thus can be easily distinguished from normal diploid wild-type plants. Further, aneuploid plants show varied flowering time and mostly have reduced fertility and seed set. Putative diploids were genotyped for at least one marker per chromosome (Chr 1: F511, CIW12; Chr 2: MSAT2.11; Chr 3: MSAT3.19, CIW11; Chr 4: nga8; Chr 5: CTR1.2, nga106). Eliminants were identified as having only C24 alleles, in addition to lacking GFP fluorescence at the centromeres which is present in the GEM line. Random diploid plants (n=8) were further confirmed by karyotyping in meiotic chromosome spreads and all were found to be diploids.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for producing a self-reproducing hybrid plant comprising:
a) obtaining a first plant comprising in its genome a first suppression cassette and a first expression cassette,
i) wherein said first suppression cassette comprises at least one first silencing element wherein said first silencing element, when expressed by said self-reproducing hybrid plant, reduces the level of at least one target sequence, wherein said target sequence comprises a member selected from the group consisting of,
A) a gene critical to meiotic second division reduction,
B) a gene critical to meiotic recombination, and
C) a gene critical to meiotic chromosome segregation,
ii) wherein said first expression cassette comprises a nucleic acid molecule encoding an active centromere-specific mutant polypeptide, wherein said active centromere-specific mutant polypeptide is expressed in said self-reproducing hybrid plant;
b) obtaining a second plant comprising in its genome a second suppression cassette and a second expression cassette,
i) wherein said second suppression cassette comprises at least one second silencing element, wherein said second silencing element, when expressed by said self-reproducing hybrid plant, reduces the level of a wild-type centromere-specific polypeptide or a homolog thereof;
ii) wherein said second expression cassette comprises a nucleic acid molecule encoding a wild-type centromere-specific polypeptide or homolog thereof, wherein said centromere-specific polypeptide is expressed in said self-reproducing hybrid plant; and
c) crossing said first plant with said second plant thereby producing said self-reproducing hybrid plant.

2. The method of claim 1, wherein said first suppression cassette comprises a silencing element with inhibitory activity against a target sequence, wherein said target sequence comprises a member selected from the group consisting of:
a) Osd1 or a homolog thereof;
b) Spo11-1 or a homolog thereof;
c) Rec8 or a homolog thereof;
d) PRD1
e) PRD2
f) PRD3
g) DYAD
h) PAIR1
i) Spo11-2

3. The method of claim 1, wherein said first expression cassette comprises a nucleic acid molecule encoding an active centromere-specific mutant polypeptide, wherein said active centromere-specific mutant polypeptide is selected from the group consisting of: CENH3-tailswap, H3.3, CENPC, MCM21, MIS12, NDC80, NUF2 and a fragment or variant of CENH3, wherein said fragment or variant is an active CENH3 mutant.

4. The method of claim 2, wherein said first expression cassette comprises a nucleic acid molecule encoding an active centromere-specific mutant polypeptide, wherein said active centromere-specific polypeptide is CENH3-tailswap.

5. The method of claim 1, wherein said first expression cassette further comprises a tissue-specific promoter operably linked to said nucleic acid molecule encoding an active centromere-specific mutant polypeptide.

6. The method of claim 4, wherein said tissue-specific promoter is an ovule-specific promoter.

7. The method of claim 6, wherein said ovule-specific promoter is the ovule-specific promoter for the BEL1 gene.

8. The method of claim 1, wherein said second suppression cassette further comprises a first inducible promoter operably linked to said second silencing element, wherein said first inducible promoter drives expression of said second silencing element in said self-reproducing hybrid plant.

9. The method of claim 8, wherein said first inducible promoter is selected from the group consisting of:
a) T7 promoter,
b) 4X UAS promoter, and
c) LexA Operator.

10. The method of claim 8, wherein said first inducible promoter is specifically induced by transactivator A;
wherein said first plant further comprises a third expression cassette comprising a first transactivator promoter operably linked to a nucleic acid molecule encoding transactivator A; and
wherein said transactivator A induces said first inducible promoter and drives expression of said second silencing element in said self-reproducing hybrid plant.

11. The method of claim 10, wherein said transactivator A is selected from the group consisting of:
a) T7 polymerase,
b) Gal4DBD-VP16, and
c) LexA-Activator fusion.

12. The method of claim 10, wherein said first transactivator promoter is an ovule-specific promoter, wherein said ovule-specific promoter drives expression of said transactivator A in the ovule of said self-reproducing hybrid plant.

13. The method of claim 12, wherein said ovule-specific promoter is the ovule-specific promoter for the BEL1 gene.

14. The method of claim 1, wherein said first suppression cassette further comprises a second inducible promoter operably linked to said first silencing element, wherein said second inducible promoter drives expression of said first silencing element in said self-reproducing hybrid plant.

15. The method of claim 12, wherein said second inducible promoter is selected from the group consisting of:
a) T7 promoter,
b) 4X UAS promoter, and
c) LexA Operator.

16. The method of claim 14, wherein said second inducible promoter is specifically induced by transactivator B,
wherein said second plant further comprises a fourth expression cassette comprising a second transactivator promoter operably linked to a nucleic acid molecule encoding transactivator B, and
wherein said transactivator B induces said second inducible promoter and drives expression of said first silencing element in said self-reproducing hybrid plant.

17. The method of claim 16, wherein said transactivator B is selected from the group consisting of:
a) T7 polymerase,
b) Gal4DBD-VP16 and
c) LexA-Activator fusion.

18. The method of claim 1, wherein said wild-type centromere-specific polypeptide is selected from the group consisting of: CENH3, CENPC, MCM21, MIS12, NDC80 and NUF2.

19. The method of claim 18, wherein said wild-type centromere-specific polypeptide is CENH3.

20. The method of claim 18, wherein said second expression cassette further comprises a tissue-specific promoter operably linked to said nucleic acid molecule encoding a wild-type centromere-specific polypeptide.

21. The method of claim 20, wherein said tissue-specific promoter is a central-cell specific promoter.

22. The method of claim 21, wherein said central cell-specific promoter is selected from the group consisting of: AT-DD7 PRO, AT-DD9 PRO, AT-DD22 PRO, AT-DD25 PRO, AT-DD36 PRO, AT-DD41 PRO, AT-DD66 PRO and AT-DD65 PRO.

23. The method claim 1, wherein said first plant is obtained by simultaneously or sequentially introducing into a plant said first suppression cassette and said first expression cassette.

24. The method of claim 1, wherein said first plant is obtained by crossing a plant comprising said first suppression cassette with a plant comprising said first expression cassette.

25. The method of claim 1, wherein said second plant is obtained by simultaneously or sequentially introducing into a plant said second suppression cassette and said second expression cassette.

26. The method of claim 1, wherein said second plant is obtained by crossing a plant comprising said second suppression cassette with a plant comprising said second expression cassette.

27. The method of claim 1, wherein said self-reproducing hybrid plant is a dicot plant.

28. The method of claim 27, wherein said dicot is Brassica, sunflower, cotton, canola, safflower, tobacco, Arabidopsis or alfalfa.

29. The method of claim 1, wherein said self-reproducing hybrid plant is soybean.

30. The method of claim 1, wherein said self-reproducing hybrid plant is a monocot plant.

31. The method of claim 30, wherein said monocot is maize, wheat, rice, barley, sorghum or rye.

32. A self-reproducing hybrid plant produced by the method of claim 1.

33. A seed of the self-reproducing hybrid plant of claim 32.

34. A suppression cassette comprising at least one first silencing element, wherein said at least one first silencing element has inhibitory activity against a target sequence, wherein said target sequence comprises a member selected from the group consisting of:
a) Osd1 or a homolog thereof;
b) Spo11-1 or a homolog thereof; and
c) Rec8 or a homolog thereof.

35. The suppression cassette of claim 34, wherein said suppression cassette comprises at least two first silencing elements wherein said at least two first silencing elements have inhibitory activity against a target sequence, wherein said target sequence comprises a member selected from the group consisting of:
a) Osd1 or a homolog thereof;
b) Spo11-1 or a homolog thereof; and
c) Rec8 or a homolog thereof.

36. The suppression cassette of claim 34, wherein said suppression cassette comprises at least three first silencing elements having inhibitory activity against target sequences:
a) Osd1 or a homolog thereof;
b) Spo11-1 or a homolog thereof; and
c) Rec8 or a homolog thereof.

37. The suppression cassette of claim 34, wherein said suppression cassette further comprises a second promoter operably linked to said at least one, at least two or at least three first silencing elements, wherein said second promoter drives expression in a plant.

38. The suppression cassette of claim 37, wherein said second promoter is an inducible promoter.

39. The suppression cassette of claim 38, wherein said second inducible promoter is specifically induced by transactivator B.

40. The suppression cassette of claim 39, wherein said second inducible promoter is selected from the group consisting of:
a) T7 promoter,
b) 4X UAS promoter and
c) LexA Operator.

41. The suppression cassette of claim 39, wherein said transactivator B is selected from the group consisting of:
a) T7 polymerase,
b) Gal4DBD-VP16 and
c) LexA-Activator fusion.

42. A suppression cassette comprising at least one silencing element, wherein said at least one silencing element has inhibitory activity against a target sequence, wherein said target sequence comprises a wild-type centromere-specific polypeptide, or a homolog thereof.

43. The suppression cassette of claim 42, wherein said wild-type centromere-specific polypeptide is selected from the group consisting of: CENH3, CENPC, MCM21, MIS12, NDC80 and NUF2.

44. The suppression cassette of claim 43, wherein said wild-type centromere-specific polypeptide is CENH3.

45. The suppression cassette of claim 43, wherein said suppression cassette further comprises a promoter operably linked to silencing element, wherein said promoter drives expression in a plant.

46. The suppression cassette of claim 42, wherein said promoter is an inducible promoter.

47. The suppression cassette of claim 46, wherein said inducible promoter is specifically induced by transactivator A.

48. The suppression cassette of claim 42, wherein said inducible promoter is selected from the group consisting of:
a) T7 promoter,
b) 4X UAS promoter and
c) LexA Operator.

49. The suppression cassette of claim 47, wherein said transactivator A is selected from the group consisting of:
a) T7 polymerase,
b) Gal4DBD-VP16 and
c) LexA-Activator fusion.

50. A plant comprising the suppression cassette of claim 34.

51. The plant of claim 50, wherein said plant is a dicot.

52. The plant of claim 51, wherein said dicot is Brassica, sunflower, cotton, canola, safflower, tobacco, Arabidopsis or alfalfa.

53. The plant of claim 52, wherein said dicot is soybean.

54. The plant of claim 50, wherein said plant is a monocot.

55. The plant of claim 54, wherein said monocot is maize, wheat, rice, barley, sorghum or rye.

56. The plant of claim 50, wherein said suppression cassette is stably incorporated into the genome of said plant.

57. An expression cassette comprising, a nucleic acid molecule comprising a nucleotide sequence encoding an active centromere-specific mutant polypeptide.

58. The expression cassette of claim 57, wherein said active centromere-specific mutant polypeptide is selected from the group consisting of: CENH3-tailswap, H3.3, CENPC, MCM21, MIS12, NDC80, NUF2 and a fragment or variant of CENH3, wherein said fragment or variant is an active CENH3 mutant.

59. The expression cassette of claim 58, wherein said active centromere-specific mutant polypeptide is CENH3-tailswap.

60. The expression cassette of claim 57, wherein said expression cassette further comprises a promoter operably linked to said nucleotide sequence, wherein said promoter drives expression in a plant.

61. The expression cassette of claim 60, wherein said promoter is an ovule-specific promoter, wherein said ovule-specific promoter drives expression of said centromere-specific polypeptide in the ovule of said plant.

62. The expression cassette of claim 61, wherein said ovule-specific promoter is the ovule-specific promoter for the BEL1 gene.

63. An expression cassette comprising, a nucleic acid molecule comprising a nucleotide sequence encoding a wild-type centromere-specific polypeptide.

64. The expression cassette of claim 63, wherein said wild-type centromere-specific polypeptide is selected from the group consisting of: CENH3, CENPC, MCM21, MIS12, NDC80 and NUF2.

65. The expression cassette of claim 64, wherein said wild-type centromere-specific polypeptide is CENH3.

66. The expression cassette of claim 65, wherein said expression cassette further comprises a promoter operably linked to said nucleic acid molecule, wherein said promoter drives expression in a plant.

67. The expression cassette of claim 66, wherein said promoter is a central cell-specific promoter, wherein said central cell-specific promoter drives expression of said wild-type centromere-specific polypeptide in the central cell of said plant.

68. The expression cassette of claim 67, wherein said central cell-specific promoter is selected from the group consisting of: AT-DD7 PRO, AT-DD9 PRO, AT-DD22 PRO, AT-DD25 PRO, AT-DD36 PRO, AT-DD41 PRO, AT-DD66 PRO and AT-65 PRO.

69. An expression cassette comprising, a nucleic acid molecule comprising a nucleotide sequence encoding transactivator A operably linked to a first transactivator promoter, wherein said first transactivator promoter drives expression of transactivator A in a plant.

70. The expression cassette of claim 69, wherein said first transactivator promoter is an ovule-specific promoter, wherein said ovule-specific transactivator promoter drives expression of said transactivator A in the ovule of said plant.

71. The expression cassette of claim 70, wherein said ovule-specific promoter is the ovule-specific promoter for the BEL1 gene.

72. The expression cassette of claim 69, wherein said transactivator A is selected from the group consisting of:
a) T7 polymerase,
b) Gal4DBD-VP16 and
c) LexA-Activator fusion.

73. An expression cassette comprising, a nucleic acid molecule comprising a nucleotide sequence encoding transactivator B operably linked to a transactivator promoter, wherein said transactivator promoter drives expression of said transactivator B in a plant.

74. The expression cassette of claim 73, wherein said transactivator promoter is selected from the group consisting of: UBI PRO, AT-EF1A PRO, GM-EF1A PRO and AT-UBIQ10 PRO.

75. The expression cassette of claim 73, wherein said transactivator B is selected from the group consisting of:
a) T7 polymerase,
b) Gal4DBD-VP16 and
c) LexA-Activator fusion.

76. A plant comprising the expression cassette of claim 57.

77. The plant of claims 76, wherein said plant is a dicot.

78. The plant of claim 77, wherein said dicot is Brassica, sunflower, cotton, canola, safflower, tobacco, Arabidopsis or alfalfa.

79. The plant of claim 78, wherein said dicot is soybean.

80. The plant of claim 76, wherein said expression cassette is stably incorporated into the genome of said plant.

81. A plant comprising stably incorporated in its genome at least one nucleic acid molecule construct selected from the group consisting of:
a) a first suppression cassette comprising at least one first silencing element having inhibitory activity against a target sequence, wherein said target sequence comprises a member selected from the group consisting of:
i) Osd1 or a homolog thereof;
ii) Spo11-1 or a homolog thereof; and
iii) Rec8 or a homolog thereof;
wherein said first silencing element is operably linked to a first inducible promoter,
wherein said first inducible promoter is induced by transactivator B;
b) a second suppression cassette comprising at least one second silencing element having inhibitory activity against a wild-type centromere-specific polypeptide, or a homolog thereof, wherein said second silencing element is operably linked to a second inducible promoter, wherein said second inducible promoter is induced by transactivator A;
c) a first expression cassette, wherein said first expression cassette comprises a first nucleic acid molecule encoding an active centromere-specific mutant polypeptide, wherein said active centromere-specific mutant polypeptide is selected from the group consisting of: CENH3-tailswap, H3.3, CENPC, MCM21, MIS12, NDC80, NUF2, and a fragment or variant of CENH3, wherein said fragment or variant is an active CENH3 mutant,
wherein said first nucleic acid molecule is operably linked to a first ovule-specific promoter, wherein activation of the first ovule-specific promoter expresses said active centromere-specific mutant polypeptide in the ovule of said plant;
d) a second expression cassette, wherein said second expression cassette comprises a second nucleic acid molecule encoding a wild-type centromere-specific polypeptide, wherein said second nucleic acid molecule is operably linked to a central-cell promoter, wherein said central cell-specific promoter expresses said wild-type centromere-specific polypeptide in the central cell of said plant;
e) a third expression cassette, wherein said third expression cassette comprises a third nucleic acid molecule encoding transactivator A, wherein said third nucleic acid molecule is operably linked to a second ovule-specific promoter, wherein said transactivator A is expressed in the ovule of said plant;
f) a fourth expression cassette, wherein said fourth expression cassette comprises a fourth nucleic acid molecule encoding transactivator B,
wherein said fourth nucleic acid molecule is operably linked to a constitutive promoter, wherein said transactivator B is expressed in said plant.

82. The plant of claim 81, wherein said plant comprises at least two nucleic acid molecule constructs comprising:
a) a first suppression cassette comprising at least one first silencing element having inhibitory activity against a target sequence, wherein said target sequence comprises a member selected from the group consisting of:
i) Osd1 or a homolog thereof;
ii) Spo11-1 or a homolog thereof; and
iii) Rec8 or a homolog thereof;
wherein said first silencing element is operably linked to a first inducible promoter, wherein said first inducible promoter is induced by transactivator B;
b) a first expression cassette, wherein said first expression cassette comprises a first nucleic acid molecule encoding an active centromere-specific mutant polypeptide, wherein said active centromere-specific mutant polypeptide is selected from the group consisting of: CENH3-tailswap, H3.3, CENPC, MCM21, MIS12, NDC80 NUF2 and a fragment or variant of CENH3, wherein said fragment or variant is an active CENH3 mutant,
wherein said first nucleic acid molecule is operably linked to a first ovule-specific promoter, wherein activation of the first ovule-specific promoter expresses said active centromere-specific mutant polypeptide in the ovule of said plant; or
c) a third expression cassette, wherein said third expression cassette comprises a third nucleic acid molecule encoding transactivator A, wherein said third nucleic acid molecule is operably linked to a second ovule-specific promoter, wherein said transactivator A is expressed in the ovule of said plant.

83. The plant of claim 82, wherein said plant comprises:
a) a first suppression cassette comprising at least one first silencing element having inhibitory activity against a target sequence, wherein said target sequence comprises a member selected from the group consisting of:
i) Osd1 or a homolog thereof;
ii) Spo11-1 or a homolog thereof; and
iii) Rec8 or a homolog thereof;
wherein said first silencing element is operably linked to a first inducible promoter, wherein said first inducible promoter is induced by transactivator B;
b) a first expression cassette, wherein said first expression cassette comprises a first nucleic acid molecule encoding an active centromere-specific mutant polypeptide, wherein said active centromere-specific mutant polypeptide is selected from the group consisting of: CENH3-tailswap, H3.3, CENPC, MCM21, MIS12, NDC80 NUF2, and a fragment or variant of CENH3, wherein said fragment or variant is an active CENH3 mutant,
wherein said first nucleic acid molecule is operably linked to a first ovule-specific promoter, wherein activation of the first ovule-specific promoter expresses said active centromere-specific mutant polypeptide in the ovule of said plant; and
c) a third expression cassette, wherein said third expression cassette comprises a third nucleic acid molecule encoding transactivator A, wherein said third nucleic acid molecule is operably linked to a second ovule-specific promoter, wherein said transactivator A is expressed in the ovule of said plant.

84. The plant of claim 81, wherein said plant comprises at least two nucleic acid molecule constructs comprising:
a) a second suppression cassette comprising at least one second silencing element having inhibitory activity against a wild-type centromere-specific polypeptide, or homolog thereof, wherein said second silencing element is operably linked to a second inducible promoter, wherein said second inducible promoter is induced by transactivator A;
b) a second expression cassette, wherein said second expression cassette comprises a second nucleic acid molecule encoding a wild-type centromere-specific polypeptide, wherein said second nucleic acid molecule is operably linked to a central-cell promoter, wherein said central cell-specific promoter expresses said wild-type centromere-specific polypeptide in the central cell of said plant and
c) a fourth expression cassette, wherein said fourth expression cassette comprises a fourth nucleic acid molecule encoding transactivator B, wherein said fourth nucleic acid molecule is operably linked to a constitutive promoter, wherein said transactivator B is expressed in said plant.

85. The plant of claim 84, wherein said plant comprises:
a) a second suppression cassette comprising at least one second silencing element having inhibitory activity against a wild-type centromere-specific polypeptide, or a homolog thereof, wherein said second silencing element is operably linked to a second inducible promoter, wherein said second inducible promoter is induced by transactivator A;
b) a second expression cassette, wherein said second expression cassette comprises a second nucleic acid molecule encoding a wild-type centromere-specific polypeptide, wherein said second nucleic acid molecule is operably linked to a central-cell promoter, wherein said central cell-specific promoter expresses said wild-type centromere-specific polypeptide in the central cell of said plant; and
c) a fourth expression cassette, wherein said fourth expression cassette comprises a fourth nucleic acid molecule encoding transactivator B, wherein said fourth nucleic acid molecule is operably linked to a constitutive promoter, wherein said transactivator B is expressed in said plant.

86. The plant of claim 77, wherein said wild-type centromere-specific polypeptide is selected from the group consisting of: CENH3, CENPC, MCM21, MIS12, NDC80, N U F2.

87. The plant of claim 86, wherein said wild-type centromere-specific polypeptide is CENH3.

88. The plant of claim 81, wherein said plant is a dicot or a monocot.

89. The plant of claim 88, wherein said dicot is Brassica, sunflower, cotton, canola, safflower, tobacco, Arabidopsis or alfalfa.

90. The plant of claim 89, wherein said dicot is soybean.

91. The plant of claim 88, wherein said monocot is maize, wheat, rice, barley, sorghum or rye.
